(19) United States
(12) Patent Application Publication
(10) Pub. No.: US 2026/0337274 A1
(43) Pub. Date: Oct. 29, 2026

(54) MULTI-DOMAIN BINDING MOLECULES

(71) Applicant: IMMUNOCORE LIMITED, Abingdon, Oxfordshire (GB)

(72) Inventors: Amandine GEORGES, Abingdon,Oxfordshire (GB); Stephen HEARTY, Abingdon,Oxfordshire (GB); Lok Hang MAK, Abingdon,Oxfordshire (GB)

(21) Appl. No.: 19/104,662

(22) PCT Filed: Aug. 18, 2023
(86) PCT No.: PCT/EP2023/072802

§ 371 Date: Feb. 18, 2025

Related U.S. Application Data
(60) Provisional application No. 63/371,861, filed on Aug. 18, 2022.

(51) Int. Cl. C07K 16/28; A61P 35/00; C07K 14/705

(52) U.S. Cl. CPC C07K 16/2833; A61P 35/00; C07K 14/7051; C07K 16/2809; C07K 2317/31; C07K 2317/52; C07K 2317/53; C07K 2317/565; C07K 2317/569; C07K 2317/622; C07K 2317/94

(57) ABSTRACT

The present invention relates to multi-domain, single-chain binding molecules. The molecules comprise i) a peptide-major histocompatibility complex (pMHC) binding domain comprising a first variable region linked to a constant region (VC1) and a second variable region linked to a constant region (VC2); ii) a T cell engaging immune effector domain comprising an antibody light chain variable region (TCE-VL) and an antibody heavy chain variable region (TCE-VH); and iii) a half-life extending domain comprising a first IgG Fc region (FC1) and a second IgG Fc region (FC2), wherein the FC1 region and FC2 region dimerise to form an Fc domain. The binding molecules can be used to treat diseases such as cancer and infectious diseases.

### SEQUENCE LISTING

The instant application contains a Sequence Listing which is hereby incorporated by reference in its entirety. Said XML copy, created on Aug. 2, 2023, is named P206512WO ST.26 Sequence Listing.xml, and is 55320 bytes in size.

### BACKGROUND TO THE INVENTION

Many protein-based therapeutics, including antibody fragments and fusion proteins, are rapidly cleared from the body following administration. Their short circulatory half-life is typically attributed to their small size, which allows for effective clearance via renal filtration, and lack of protection from intracellular degradation. In such cases, frequent administration or long infusion times are required to maintain an effective concentration of the drug over prolonged periods. To improve dosing, several strategies have been employed to extend circulatory half-life. These include increasing the hydrodynamic radius of the protein through attachment of flexible hydrophilic molecules, such as carbohydrate or PEG (polyethelene glycol), and exploiting recycling via the neonatal Fc receptor (FcRn), through attachment of antibody Fc domains or serum albumin (Konnteman, Curr Opin Biotechnol. 2011 December; 22(6):868-76).

Strategies that exploit FcRn mediated recycling are particularly attractive because of the lower risk of inducing immunogenicity in vivo and long half-life extensions that may be achieved. For example, the half-life of a T cell engaging bispecific antibody of the BiTE® format, is reported to be in excess of 200 h, following attachment of an Fc domain (Lorenczewski, et al., Blood 2017. 130(Suppl 1), 2815). Similarly, bispecific antibodies of the TriTac® format, which incorporate an albumin binding domain, are reported to have a half-life of over four days (Wesche et al., Cancer Res 2018; 78(13 Suppl):Abstract nr 3814).

Fusion proteins comprising a soluble T cell receptor (TCR) fused to an anti-CD3 antibody fragment are a relatively new category of immune cell (e.g., T cell) engaging bispecific fusion proteins, with an in vivo half-life in the region of 6-8 h (Sato et al., 2018 J Clin Onc 2018 36, no. 15, suppl 9521-9521; Middleton et al., J Clin Onc 2016 34, no. 15, suppl 3016-3016). This is far shorter than traditional monoclonal antibodies, which typically have a half-life in the range of 260-720 hours (Ovacik & Lin, 2018 Clin Transl Sci, 11:540). Furthermore TCR-anti-CD3 fusion proteins have demonstrated advantageous therapeutic properties including picomolar potency (Lowe et al. 2019 Cancer treatment reviews, vol. 77 35-43). There is therefore a need to identify suitable approaches for extending the half-life of TCR-immune cell engaging domain fusion proteins, such as TCR-anti-CD3 fusion proteins, and other TCR-containing proteins in order to reduce dosing frequency and maintain effective concentrations over a prolonged period of time, without impacting other therapeutic properties.

Unlike traditional antibodies, TCRs are designed to recognize short peptides derived from intracellular antigens and presented on the cell surface by human leukocyte antigen (peptide-HLA). Effective immune synapse formation between a peptide-HLA complex on an antigen presenting cell and the corresponding receptor on an immune cell such as a T cell relies on carefully choreographed interactions which can be perturbed by increases in intermembrane distance, for example (Choudhuri et al., 2005 Nature July 28; 436(7050):578-82; Holland et al J Clin Invest. 2020; 130(5):2673-2688). Therefore, fusion approaches for increasing the half-life of TCR-containing proteins, such as attachment of antibody Fc domains or serum albumin, are highly challenging due to the risk of perturbing the interaction geometry required for TCR binding. Similar challenges also apply to fusion proteins containing antibodies that bind to peptide-HLA complexes, which are known as TCR-like or TCR-mimic antibodies.

WO 2020/157211 describes an approach for extending the half-life of a TCR-anti-CD3 fusion protein by fusing it to an immunoglobulin Fc domain or an albumin-binding domain. However, such multi-domain binding molecules are large and complex proteins, for which there are a myriad of possible formats, i.e., possible combinations of positions and orientations of each domain (and each region in each domain) on one or more polypeptide chains. The position and orientation of each domain (and regions thereof) in the molecule, and the number of polypeptide chains present, can influence characteristics of the binding molecule such as activity, half-life and manufacturability. Thus, there remains a need to identify favorable formats for such multi-domain binding molecules.

### SUMMARY OF THE INVENTION

The present invention relates generally to multi-domain binding molecules. The invention particularly relates to multi-domain binding molecules that comprise i) a peptide-major histocompatibility complex (pMHC) binding domain comprising a first variable region linked to a constant region (VC1) and a second variable region linked to a constant region (VC2); ii) an immune cell engaging (ICE) domain (such as a T cell engaging immune effector domaincomprising, for example, an antibody light chain variable region (TCE-VL) and an antibody heavy chain variable region (TCE-VH); and iii) a half-life extending domain comprising a first IgG Fc region (FC1) and a second IgG Fc region (FC2), wherein the FC1 region and FC2 region dimerise to form an Fc domain. The binding molecules can be used to treat diseases such as cancer, infectious diseases and autoimmune diseases.

### DESCRIPTION OF THE INVENTION

The inventors tested over 35 different formats (i.e., orientations and positions of each domain in the polypeptide) for a multi-domain binding molecule comprising a pMHC binding domain, a T cell engaging immune effector domain and a half-life extending domain. In doing so, they found that, in many formats, fusing a TCR-anti-CD3 fusion protein to an Fc domain resulted in a substantial loss of potency in vitro. However, the inventors surprisingly identified a format for such a molecule which can be expressed as a single polypeptide chain, has a significantly enhanced half-life, and which retains the high potency of the original molecule. Example 8 further shows that the identified format works advantageously with TCRs binding to different targets.

In a first aspect, there is provided a multi-domain, single-chain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) a an immune cell engaging (ICE) domain; and
iii) a half-life extending domain comprising a first IgG Fc region (FC1) and a second IgG Fc region (FC2), wherein the FC1 region and FC2 region dimerise to form an Fc domain;
wherein the ICE domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of the FC2 region; and
wherein the pMHC binding domain and the T cell engaging immune effector domain are capable of binding to a pMHC complex and an immune cell respectively.

In one embodiment, there is provided a multi-domain, single-chain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) a T cell engaging immune effector domain comprising an antibody light chain variable region (TCE-VL) and an antibody heavy chain variable region (TCE-VH); and
iii) a half-life extending domain comprising a first IgG Fc region (FC1) and a second IgG Fc region (FC2), wherein the FC1 region and FC2 region dimerise to form an Fc domain;
wherein the T cell engaging immune effector domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of the FC2 region; and
wherein the pMHC binding domain and the T cell engaging immune effector domain are capable of binding to a pMHC complex and a T cell respectively.

In another aspect, there is provided a multi-domain, single-chain binding molecule comprising:
i) a soluble TCR comprising a first variable region linked to a constant region (VC1) and a second variable region linked to a constant region (VC2), wherein VC1 comprises a TCRβ variable and constant region having the amino acid sequence provided in SEQ ID NO: 16, or a sequence that is at least 90%, at least 95%, or at least 98% identical thereto, and VC2 comprises a TCRα variable and constant region having the amino acid sequence provided in SEQ ID NO: 14, or a sequence that is at least 90%, at least 95%, or at least 98% identical thereto;
ii) an anti-CD3 scFv comprising an antibody light chain variable region (TCE-VL) having the amino acid sequence provided in SEQ ID NO: 31, or a sequence that is at least 90%, at least 95%, or at least 98% identical thereto, and an antibody heavy chain variable region (TCE-VH) having the amino acid sequence provided in SEQ ID NO: 32, or a sequence that is at least 90%, at least 95%, or at least 98% identical thereto; and
iii) a half-life extending domain comprising a first IgG Fc region (FC1) having the amino acid sequence provided in SEQ ID NO: 42, or a sequence that is at least 90%, at least 95%, or at least 98% identical thereto, and a second IgG Fc region (FC2) having the amino acid sequence provided in SEQ ID NO: 43, or a sequence that is at least 90%, at least 95%, or at least 98% identical thereto, wherein the FC1 region and FC2 region dimerise to form an Fc domain;
wherein the T cell engaging immune effector domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of the FC2 region; and
wherein the pMHC binding domain and the T cell engaging immune effector domain are capable of binding to a pMHC complex and a T cell respectively.

In another aspect, there is provided a multi-domain, single-chain binding molecule comprising the amino acid sequence provided in SEQ ID NO: 45.

In yet a further aspect, there is provided a nucleic acid encoding the multi-domain binding molecule. There is also provided an expression vector comprising the nucleic acid of this aspect. In addition, there is provided a host cell comprising the nucleic acid or the vector of this aspect.

Also provided, in a further aspect, is a method of making the multi-domain binding molecule, comprising maintaining the host cell described above under optimal conditions for expression of the nucleic acid and isolating the multi-domain binding molecule.

In a further aspect, there is provided a pharmaceutical composition comprising the multi-domain binding molecule.

The multi-domain binding molecule, the nucleic acid, the vector, the host cell or the pharmaceutical composition of any of the above aspects may be used in the treatment of diseases such as cancer, infectious diseases and autoimmune diseases. Thus, in a further aspect, also provided is the multi-domain binding molecule, the nucleic acid, the vector, the host cell or the pharmaceutical composition for use as a medicament. In a still further aspect there is provided a method of treatment comprising administering the multi-domain binding molecule, the nucleic acid, the vector, the host cell or the pharmaceutical composition to a patient in need thereof.

Peptide-Major Histocompatibility Complex (pMHC) Binding Domains

A “pMHC binding domain”, as used herein, is a protein domain capable of binding to a peptide-MHC complex. A first variable region linked to a constant region (VC1) and a second variable region linked to a constant region (VC2) dimerise to form the pMHC binding domain. In this context, “VC1” refers to a region of the pMHC binding domain sequence that comprises the first variable region linked to a constant region and “VC2” refers to a region that comprises the second variable region linked to a constant region. The pMHC binding site is within the variable regions of VC1 and VC2. Suitable variable and constant region sequences include TCR or antibody variable and constant regions. The terms “MHC” and “HLA” as used herein are used interchangeably.

The pMHC binding domain may comprise at least part of a TCRα and a TCRβ chain. For example, the variable regions of VC1 and VC2 may be TCR variable regions. VC1 may comprise either a TCRα or a TCRβ variable region and VC2 may comprise the other of the TCRα and TCRβ variable regions.

For example:
(i) VC1 may comprise either (a) a TCRα variable and constant region or (b) a TCRβ variable and constant region; and
(ii) VC2 may comprise the other of (a) or (b). Preferably, VC1 comprises the TCRβ variable and constant region and VC2 comprises the TCRα variable and constant region.

The pMHC binding domain may be a T cell receptor (TCR), such as a soluble TCR, comprising TCR variable regions and constant regions. The TCR sequences defined herein are described with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011 (6): 595-603; Lefranc, (2001), Curr Protoc Immunol Appendix 1: Appendix 100; and Lefranc, (2003), Leukemia 17 (1): 260-266. Briefly, TCRs consist of two disulfide linked chains. Each chain (alpha and beta) is generally regarded as having two extracellular regions, namely a variable and a constant region. A short joining region connects the variable and constant regions and is typically considered part of the alpha variable region. Additionally, the beta chain usually contains a short diversity region next to the joining region, which is also typically considered part of the beta variable region. The variable region of each chain of a typical TCR is located N-terminally and comprises three Complementarity Determining Regions (CDRs) embedded in a framework sequence. The CDRs comprise the recognition site for peptide-MHC binding.

Alternatively, the pMHC binding domain may comprise variable regions of an antibody. The VC1 and VC2 variable regions may be antibody heavy or light chain variable regions. For example, VC1 may comprise either a heavy or a light chain antibody variable region and VC2 may comprise the other of the heavy or a light chain antibody variable region. In this regard, the pMHC binding domain may be a TCR-like antibody, also known as a “TCR mimic antibody” (TCRm-Ab). For example, the pMHC binding domain may comprise variable regions of a TCR-like antibody. Antibodies do not naturally recognize a pMHC complex. However, it is known that antibodies with specificity for pMHC can be engineered, as described in Chang et al., Expert Opin Biol Ther. 2016 August; 16(8):979-87 and Dahan et al., Expert Rev Mol Med. 2012 Feb. 24; 14:e6.

The pMHC binding domain may comprise at least one immunoglobulin constant region. For example the constant regions in VC1 and VC2 may be immunoglobulin constant regions. The constant region may correspond to a constant region from a TCRα chain or a TCRβ chain (TRAC or TRBC respectively). Alternatively the constant regions of the pMHC binding domain may be a constant region from an antibody light or heavy chain (CL, CH1, CH2, CH3 or CH4). The constant region may be full length or may be truncated. TCR constant regions may be truncated to remove the transmembrane domain and cytoplasmic tail. Where the constant region is truncated, preferably only membrane-associated and cytoplasmic portions are removed from the C-terminal end. Where the pMHC binding domain comprises TCRα or TCRβ chain sequences, VC1 and VC2 may each comprise a TCR variable region and a TCR constant region. Preferably, VC1 and VC2 do not comprise a transmembrane or cytoplasmic domain, i.e., preferably the pMHC binding domain is soluble. Additional mutations may be introduced into the amino acid sequence of the constant regions relative to natural constant regions. The constant regions may also include residues, either naturally-occurring or introduced, that allow for dimerisation by, for example, a disulphide bond between two cysteine residues.

If present, TCR portions of the molecules of the invention may be αβ heterodimers. Alpha-beta heterodimeric TCR portions of the molecules of the invention may comprise an alpha chain TRAC constant region sequence and/or a beta chain TRBC1 or TRBC2 constant region sequence. As described above, the constant regions may be in soluble format (i.e. having no transmembrane or cytoplasmic domains). One or both of the constant regions may contain mutations, substitutions or deletions relative to the native TRAC and/or TRBC1/2 sequences. The terms TRAC and TRBC1/2 also encompass natural polymorphic variants, for example N to K at position 4 of TRAC (Bragado et al International immunology. 1994 February; 6 (2): 223-30).

Alpha and beta chain constant region sequences may be modified by truncation or substitution to delete the native disulphide bond between Cys4 of exon 2 of TRAC and Cys2 of exon 2 of TRBC1 or TRBC2. Alpha and/or beta chain constant region sequence(s) may have an introduced disulphide bond between residues of the respective constant domains, as described, for example, in WO 2003/020763, WO 2004/033685 and WO 2006/000830, and for example, in U.S. Pat. Nos. 7,329,731, 7,569,664; and 8,361,794, the contents of each of which are herein incorporated by reference. Alpha and beta constant regions may be modified by substitution of cysteine residues at position Thr 48 of TRAC and position Ser 57 of TRBC1 or TRBC2, the said cysteines forming a disulphide bond between the alpha and beta constant regions of the TCR. TRBC1 or TRBC2 may additionally include a cysteine to alanine mutation at position 75 of the constant domain and an asparagine to aspartic acid mutation at position 89 of the constant domain. One or both of the extracellular constant regions present in an αβ heterodimer may be truncated at the C terminus or C termini, for example by up to 15, or up to 10, or up to 8 or fewer amino acids. The C terminus of an alpha chain extracellular constant region may be truncated by 8 amino acids.

The amino acid sequence of the VC1 and VC2 variable and constant regions may correspond to those found in nature, or they may contain one or more mutations relative to a natural protein. Such mutations may be made to increase the affinity of the pMHC binding domain for a given antigen.

Additionally or alternatively, mutations may be incorporated to improve stability and manufacturability. The VC1 and VC2 sequences may be derived from human sequences.

The VC1 and VC2 sequences may comprise one or more engineered cysteine residues in the constant region to form a non-native disulphide bond between VC1 and VC2. Suitable positions for introducing disulphide bond between residues of the respective constant regions, are described in WO 2003/020763 and WO 2004/033685. Single chain TCRs are further described in WO2004/033685; WO98/39482; WO01/62908; Weidanz et al. (1998) J Immunol Methods 221 (1-2): 59-76; Hoo et al. (1992) Proc Natl Acad Sci USA 89 (10): 4759-4763; Schodin (1996) Mol Immunol 33 (9): 819-829).

The VC1 may comprise a TCRα or TCRβ variable region and VC2 may comprise the other of the TCRα and TCRβ variable region. Preferably:
(i) the TCRα variable region comprises CDRs of SEQ ID NO: 3, 4, and 5 as CDR1, CDR2 and CDR3 respectively; and
(ii) the TCRβ variable region comprises CDRs of SEQ ID NO: 9, 10, and 11 as CDR1, CDR2 and CDR3 respectively.

Alternatively, the TCRα and TCRβ CDR sequences may each optionally have one, two, three, or four amino acid substitutions relative to the sequences recited above.

The TCRα variable region may comprise CDRs that are at least 90%, at least 95%, at least 98%, or at least 99% identical to the sequence of SEQ ID NO: 3, 4, and 5 as CDR1, CDR2 and CDR3 and/or the TCRβ variable region may comprise CDRs that are at least 90%, at least 95%, at least 98%, or at least 99% identical to SEQ ID NO: 9, 10, and 11 as CDR1, CDR2 and CDR3 respectively.

The TCRα variable region may comprise CDRs that correspond to the sequences of SEQ ID NO: 3, 4, and 5, and comprise FRs that are at least 90%, at least 95%, at least 98%, or at least 99% identical to the sequences of SEQ ID NO: 27, 6, 7 and 28, and/or the TCRβ variable region may comprise CDRs that correspond to the sequences of SEQ ID NO: 9, 10, and 11, and comprise FRs that are at least 90%, at least 95%, at least 98%, or at least 99% identical to the sequences of SEQ ID NO: 29, 12, 13 and 30.

The TCRα variable region may be at least 80% identical to the sequence of SEQ ID NO: 2 and the TCRβ variable region may be at least 80% identical to the sequence of SEQ ID NO: 8. The TCRα variable region may be at least 90%, at least 95%, or at least 98% identical to the sequence of SEQ ID NO: 2 and the TCRβ variable region may be at least 90%, at least 95%, or at least 98% identical to the sequence of SEQ ID NO: 8. Preferably, the TCRα variable region has the sequence provided in SEQ ID NO: 2 and the TCRβ variable region has the sequence provided in SEQ ID NO: 8.

VC1 may comprise a TCRα or TCRβ constant region and VC2 may comprise the other of the TCRα and TCRβ constant region. The TCRα constant region may be at least 80% identical to the sequence of SEQ ID NO: 15 and the TCRβ constant region may be at least 80% identical to the sequence of SEQ ID NO: 19. The TCRα constant region may be at least 90%, at least 95%, or at least 98% identical to the sequence of SEQ ID NO: 15 and the TCRβ constant region may be at least 90%, at least 95%, or at least 98% identical to the sequence of SEQ ID NO: 19. Preferably, the TCRα constant region has the sequence provided in SEQ ID NO: 15 and the TCRβ constant region has the sequence provided in SEQ ID NO: 19.

VC1 may comprise a TCRα variable and constant region or TCRβ variable and constant region and VC2 may comprise the other of the TCRα and TCRβ variable and constant regions. The TCRα variable and constant region may comprise, or consist of, an amino acid sequence that is at least 80% identical to the sequence of SEQ ID NO: 14 and the TCRβ variable and constant region may comprise, or consist of, an amino acid sequence that is at least 80% identical to the sequence of SEQ ID NO: 16. The TCRα variable and constant region may comprise, or consist of, an amino acid sequence that is at least 90%, at least 95%, or at least 98% identical to the sequence of SEQ ID NO: 14 and the TCRβ variable and constant region may comprise, or consist of, an amino acid sequence that is at least 90%, at least 95%, or at least 98% identical to the sequence of SEQ ID NO: 16. Preferably, the TCRα variable and constant region comprises, or consists of, the amino acid sequence provided in SEQ ID NO: 14 and the TCRβ variable and constant region comprises, or consists of, the amino acid sequence provided in SEQ ID NO: 16.

The skilled person would appreciate that the format of the multi-domain binding molecule of the invention could equally be applied TCR sequences other than those recited above. For example, other suitable TCR chain amino acid sequences are provided in WO2011001152, WO2017109496, WO2017175006 and WO2018234319, and, for example, in U.S. Pat. Nos. 8,519,100, 11,639,374, 11,505,590, and 11,427,624, the contents of each which are herein incorporated by reference.

As is well-known in the art, protein molecules may be subject to post-translational modifications. Glycosylation is one such modification, which comprises the covalent attachment of oligosaccharide moieties to defined amino acids in a TCR or antibody chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34). Glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci.August; 94(8):1626-35). Alternatively, glycosylation can lead to a lack of consistency in manufacturing which is not desirable for a therapeutic molecule. Residues at high risk of glycosylation, such as asparagine, may be substituted with an alternative amino acid, such as glutamine.

VC1 and/or VC2 may comprise one or more amino acid substitutions which remove one or more glycosylation sites. The substitutions in this context are relative to a native (e.g., wild-type) sequence or unmodified sequence. For example:
(i) VC1 or VC2 may comprise a TCRα variable and constant region comprising one or more amino acid substitutions at positions selected from the group consisting of N24, N148, N182 and N193, numbered according to SEQ ID NO: 14; and/or
(ii) the other of VC1 and VC2 may comprise a TCRβ variable and constant region comprising an amino acid substitution at position N184, numbered according to SEQ ID NO: 16. The substitutions may be Asn to Gln (i.e., N to Q) substitutions. Preferably, the TCRα variable and constant region comprises N24Q, N148Q, N182Q and N193Q substitutions, numbered according to SEQ ID NO: 14, and the TCRβ variable and constant region comprises a N184Q substitution, numbered according to SEQ ID NO: 16.

The pMHC binding domain may not be fully aglycosylated, i.e., the pMHC may retain one or more glycosylation site(s) from its native sequence. For example, the pMHC binding domain may be glycosylated at a single glycosylation site (i.e., the pMHC binding domain may contain only one glycosylation site). The single glycosylation site may be in the variable region of VC1 or VC2. The single glycosylation site may be at position N18 of a TCRβ variable region, numbered according to SEQ ID NO: 16. Advantageously, the present inventors have identified that multi-domain binding proteins with this single glycosylated site have better manufacturability (e.g., protein production yield, resistance to thermal stress and aggregation), as compared to other glycosylated and/or aglycosylated variants, in addition to retaining affinity for peptide-MHC binding and potency of target cell killing.

The pMHC binding domain binds to MHC in complex with a peptide antigen. The peptide antigen may be a disease associated antigen. The pMHC binding domain may bind to a tumour associated antigen peptide in complex with an MHC. For example, the peptide antigen may be a peptide derived from GP100, NYESO, MAGEA4, or PRAME as described in WO2011001152, WO2017109496, WO2017175006 and WO2018234319. The tumour associated antigen may be PIWIL1. For example, the pMHC binding domain may bind to a SLSNRLYYL (SEQ ID NO: 56) HLA-A*02 complex. The tumour associated antigen may be PRAME. Preferably, the pMHC binding domain binds to a SLLQHLIGL (SEQ ID NO: 1) HLA-A*02 complex.

### Immune Cell Engaging Domains

An “Immune cell engaging domain”, as used herein, is a protein domain that is capable of binding to a target on an immune cell and/or modifying an immune response, for example promoting or suppressing an immune response such as T cell activation. The immune cell engaging domain is also referred to herein as the “ICE” domain.

In some embodiments, the immune cell engaging domain comprises an antibody light chain variable region (ICE-VL) and an antibody heavy chain variable region (ICE-VH). As used herein, “ICE-VL” and “ICE-VH” refer to the light chain variable region and the heavy chain variable region of the immune cell engaging domain, respectively. “ICE-VL” and “ICE-VH” may also be referred to as “ICEVL” and “ICEVH” herein. Thus, the immune cell engaging domain may comprise an antigen-binding site. The antibody may also be a single domain antibody (“ICE-SD”), such as the variable region of a heavy chain antibody (e.g., a VHH).

The immune cell engaging domain may be a T cell engaging immune effector domain. A “T cell engaging immune effector domain”, as used herein, is a protein domain that is capable of binding to a target on a T cell to promote an immune response. The T cell engaging immune effector domain may comprise an antibody light chain variable region (TCE-VL) and an antibody heavy chain variable region (TCE-VH). As used herein, “TCE-VL” and “TCE-VH” refer to the light chain variable region and the heavy chain variable region of the T cell engaging immune effector domain. “TCE-VL” and “TCE-VH” may also be referred to as “TCEVL” and “TCEVH” herein.

The T cell engaging immune effector domain may bind to a protein expressed on a cell surface of a T cell to promote activation of the T cell. For example, the T cell engaging immune effector domain may be a CD3 effector domain. The T cell engaging immune effector domain may bind to, for example specifically bind to, CD3 (i.e., the T cell engaging immune effector domain may be a CD3-binding protein). The T cell engaging immune effector may be an antibody, or a functional fragment thereof, for example a single-chain variable fragment (scFv), or a similar sized antibody-like scaffold, or any other binding protein that activates a T cell through interaction with CD3 and/or the TCR/CD3 complex. The antibody may also be a single domain antibody, such as the variable region of a heavy chain antibody (e.g., a VHH).

Alternatively, the immune cell engaging domain may be an immune suppressor. As used herein, the term “immune suppressor” refers to any molecule, e.g., a protein, that is capable of inhibiting an immune response, such as inhibiting T cell activation. The immune suppressor may bind to a target (e.g. antigen). For example, the immune suppressor may be an immune checkpoint agonist, i.e., a molecule that induces immune checkpoint signalling. The immune suppressor may comprise an antigen-binding moiety that is capable of binding to an antigen. The antigen of the immune suppressor may be located on an immune cell, such as a T cell. The binding molecule may comprise an antibody or antigen binding fragment thereof, for example, the antibody may also be a single domain antibody, such as the variable region of a heavy chain antibody. Alternatively, the antibody may be a single-chain variable fragment (scFv), or a similar sized antibody-like scaffold, or any other binding protein that suppresses a T cell through induction of immune checkpoint signalling. Such immune suppressors are described below.

The immune cell engaging domain may comprise an antigen-binding moiety that is capable of binding to an antigen. The antigen of the immune cell engaging domain may be located on an immune cell, such as a T cell. The binding molecule may comprise an antibody or antigen binding fragment thereof.

The term “antibody” as used herein is meant to include conventional/native antibodies and engineered antibodies, in particular functional antibody fragments, single chain antibodies, single domain antibodies, and bispecific or multispecific antibodies. “Native” or “conventional”, in this context, refers to an antibody that has the same type of domains and domain arrangements as an antibody found in nature and comprises antibody-derived CDR and FR sequences. In a native/conventional four-chain, e.g. human, antibody, two heavy chains are linked to each other by disulfide bonds and each heavy chain is linked to a light chain by a disulfide bond. The variable domains of both light (VL) and heavy (VH) chains determine binding recognition and specificity to the antigen. Conventional antibody binding sites are made up of residues that are primarily from the “antibody complementarity determining regions” (CDRs) or hypervariable regions. Occasionally, residues from non-hypervariable or framework regions (FR) influence the overall domain structure and hence the binding site. CDRs refer to amino acid sequences that together define the binding affinity and specificity of the native antibody binding site. The light and heavy chains of a conventional four-chain antibody each have three CDRs, designated CDR1-L, CDR2-L, CDR3-L and CDR1-H, CDR2-H, CDR3-H, respectively. A conventional four-chain antibody antigen-binding site, therefore, includes six CDRs, comprising the CDR set from each of a VH and VL.

“Engineered” antibody formats include functional antibody fragments, single chain antibodies, single domain antibodies, and chimeric, humanized, bispecific or multispecific antibodies. Engineered antibody formats further include constructs in which TCR-derived CDRs, possibly including additional 3, 2 or 1 N and/or C terminal framework residues, or entire TCR-derived variable domains are grafted onto antibody heavy or light chains. A “functional antibody fragment” refers to a portion of a full-length antibody, or a protein that resembles a portion of a full-length antibody, that retains the ability to bind to its target antigen, in particular the antigen binding region or variable region of the full-length antibody. Examples of functional antibody “fragments” include Fv, Fab, F(ab′)2, Fab′, dsFv, (dsFv)2, scFv, sc(Fv)2 and diabodies. For example, a binding molecule of the invention may comprise a scFv.

The antibody may also be a single domain antibody, such as the variable region of a heavy chain antibody. In this regard, the term “single domain antibody” refers to an antibody that consists of a single antibody variable domain (e.g., a heavy chain variable domain). Thus, the immune cell engaging domain may comprise a VHH (i.e., the variable domain of a heavy chain antibody), for example. As is known in the art, the antigen binding site of a single domain antibody, such as a VHH, may comprise three CDRs (as opposed to six in a conventional four-chain antibody). The term “antigen binding moiety of an antibody”, as used herein, encompasses such binding sites. Alternatively, or additionally, the binding molecule may comprise a Fab or Fv fragment. The term “Fab” (“fragment antigen-binding”) denotes an antigen-binding fragment of an antibody, which comprises the antibody light chain (VL-CL) and the variable and CH1 domain (VH—CH1) of the antibody heavy chain. Fab fragments typically have a molecular weight of about 50,000 Dalton. The Fv fragment is the N-terminal part of the Fab fragment of an antibody and consists of the variable portions of one light chain (VL) and one heavy chain (VH).

The immune cell engaging domain may comprise an antibody heavy chain variable region (VH) and an antibody light chain variable region (VL), which associate to form the antigen-binding moiety that is capable of binding to the antigen. Thus, the antigen binding moiety may comprise the VH and the VL. For example, the immune cell engaging domain may comprise a scFv comprising the VH and VL.

Other suitable antigen binding moieties are heavy chain antibodies (hcAb), single domain antibodies (sdAb), minibodies (Tramontano et al (1994) J. Mol. Recognition 7, 9-24), the variable domain of camelid heavy chain antibodies (VHH), the variable domain of the new antigen receptors (VNAR), affibodies (Nygren P.A. (2008) FEBS J. 275, 2668-2676), alphabodies (see WO2010066740), designed ankyrin-repeat domains (DARPins) (Stumpp et al (2008) Drug Discovery Today 13, 695-701), anticalins (Skerra et al (2008) FEBS J. 275, 2677-2683), knottins (Kolmar et al (2008) FEBS J. 275, 2684-2690) and engineered CH2 domains (nanoantibodies, see Dimitrov DS (2009) mAbs 1, 26-28).

The antigen binding moiety may be, or comprise, a heavy chain variable domain that comprises, consists or essentially consists of four framework regions (FR1 to FR4 respectively) and three complementarity determining regions (CDR1 to CDR3 respectively); or any suitable fragment of such a heavy chain variable domain (which retains the antigen binding site). The antigen binding moiety may be a heavy chain antibody. The antigen binding moiety may be a heavy chain variable domain sequence of an antibody that is derived from a conventional four-chain antibody, such as, without limitation, a VH sequence that is derived from a human antibody. Preferably, the antigen binding moiety is, or comprises, the variable domain of a heavy chain antibody (e.g., a camelid antibody), such as a VHH (also referred to herein as a “VHH domain”). Preferably, the antigen binding moiety is a VHH.

As described herein, the immune cell engaging domain may comprise an antigen binding moiety (e.g., an antibody antigen binding moiety) that binds to an antigen located on an immune cell. In the context of the present invention, “immune cell” may refer to, for example, a T cell or a B cell. In particular, the antigen of the antigen-binding moiety may be a T cell surface antigen.

The immune cell engaging domain may be a single-chain variable fragment (scFv). “Single-chain Fv” also abbreviated as “sFv” or “scFv” are antibody fragments that comprise the VH and VL antibody domains connected into a single polypeptide chain. The scFv polypeptide may further comprise a polypeptide linker between the VH and VL domains which enables the scFv to form the desired structure for antigen binding. For a review of scFv's, see Pluckthun in The Pharmacology of

Monoclonal Antibodies, vol. 113, Rosenburg and Moore eds., Springer-Verlag, New York, pp. 269-315 (1994).

The immune cell engaging domain may be a CD3 effector. CD3 effectors include but are not limited to anti-CD3 antibodies or antibody fragments, in particular an anti-CD3 scFv or antibody-like scaffolds.

The immune cell engaging domain may be a T cell engaging immune effector domain which may be an anti-CD3 scFv. Further immune effectors include but are not limited to antibodies, including fragments, derivatives and variants thereof, that bind to antigens on T cells. Such antigens include CD28, 4-1bb (CD137) or CD16 or any molecules that exert an effect at the immune synapse. A particularly preferred immune effector is an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, Fab fragments, F(ab′) 2 fragments, dsFv and scFv fragments.

Preferably, the immune cell engaging domain is a T cell engaging immune effector domain comprising:
(i) a VL region comprising CDRs of SEQ ID NO: 33, 34, and 35 as CDR1, CDR2 and CDR3 respectively; and
(ii) a VH region comprising CDRs of SEQ ID NO: 36, 37, and 38 as CDR1, CDR2 and CDR3 respectively.

Alternatively, the immune cell engaging domain may be a T cell engaging immune effector domain comprising:
(i) a VL region comprising CDRs of SEQ ID NO: 33, 34, and 35 as CDR1, CDR2 and CDR3 respectively; and
(ii) a VH region comprising CDRs of SEQ ID NO: 48, 37, and 38 as CDR1, CDR2 and CDR3 respectively.

The VL and VH CDR sequences above may each optionally have one, two, three, or four amino acid substitutions relative to the sequences recited above.

The TCE-VL may comprise CDRs that are at least 90%, at least 95%, at least 98%, or at least 99% identical to the sequence of SEQ ID NO: 33, 34, and 35 as CDR1, CDR2 and CDR3 and/or the TCE-VH may comprise CDRs that are at least 90%, at least 95%, at least 98%, or at least 99% identical to SEQ ID NO: 36, 37, and 38 as CDR1, CDR2 and CDR3 respectively.

Alternatively, the TCE-VL may comprise CDRs that are at least 90%, at least 95%, at least 98%, or at least 99% identical to the sequence of SEQ ID NO: 33, 34, and 35 as CDR1, CDR2 and CDR3 and/or the TCE-VH may comprise CDRs that are at least 90%, at least 95%, at least 98%, or at least 99% identical to SEQ ID NO: 48, 37, and 38 as CDR1, CDR2 and CDR3 respectively.

The TCE-VL may comprise, or consist of, an amino acid sequence that is at least 80% identical to the sequence of SEQ ID NO: 31 and the TCE-VH may comprise, or consist of, an amino acid sequence that is at least 80% identical to the sequence of SEQ ID NO: 32. The TCE-VL may comprise, or consist of, an amino acid sequence that is at least 90%, at least 95%, or at least 98% identical to the sequence of SEQ ID NO: 31 and the TCE-VH may comprise, or consist of, an amino acid sequence that is at least 90%, at least 95%, or at least 98% identical to the sequence of SEQ ID NO: 32. Preferably, the TCE-VL comprises, or consists of, the amino acid sequence provided in SEQ ID NO: 31 and the TCE-VH comprises, or consists of, the amino acid sequence provided in SEQ ID NO: 32.

Alternatively, the TCE-VL comprises, or consists of, an amino acid sequence that is at least 80% identical to the sequence of SEQ ID NO: 31 and the TCE-VH comprises, or consists of, an amino acid sequence that is at least 80% identical to the sequence of SEQ ID NO: 41. The TCE-VL may comprise, or consist of, an amino acid sequence that is at least 90%, at least 95%, or at least 98% identical to the sequence of SEQ ID NO: 31 and the TCE-VH may comprise, or consist of, an amino acid sequence that is at least 90%, at least 95%, or at least 98% identical to the sequence of SEQ ID NO: 41. For example, the TCE-VL may comprise, or consist of, the amino acid sequence provided in SEQ ID NO: 31 and the TCE-VH may comprise, or consist of, the amino acid sequence provided in SEQ ID NO: 41.

As described above, the immune cell engaging domain or T cell engaging immune effector domain may be an scFv. The immune cell engaging domain or T cell engaging immune effector domain may be an scFv comprising, or consisting of, an amino acid sequence that is at least 80% identical to the sequence of SEQ ID NO: 17 or 40. The scFv may comprise, or consist of, an amino acid sequence that is at least 90%, at least 95%, or at least 98% identical to the sequence of SEQ ID NO: 17 or 40. Preferably, the scFv comprises, or consists of, the amino acid sequence provided in SEQ ID NO: 17. Alternatively, the scFv may comprise, or consist of, the amino acid sequence provided in SEQ ID NO: 40.

The immune cell engaging domain may alternatively be an immune suppressor. For example, the target of the immune suppressor may be an immune checkpoint molecule, such as PD-1 (Programmed Death 1 receptor), A2AR (Adenosine A2A receptor), A2BR (Adenosine A2B receptor), B7-H3 (B7 Homolog 3, also called CD276) B7-H4 (B7 Homolog 4, also called VTCN1), BTLA (B and T Lymphocyte Attenuator, also called CD272), CTLA-4 (Cytotoxic T-Lymphocyte-Associated protein 4, also called CD152), IDO (Indoleamine 2,3-dioxygenase), CD200 Receptor, KIR (Killer-cell Immunoglobulin-like Receptor), TIGIT (T cell Immunoreceptor with Ig and ITIM domains), LAG3 (Lymphocyte Activation Gene-3), NOX2 (nicotinamide adenine dinucleotide phosphate NADPH oxidase isoform 2), TIM-3 (T-cell Immunoglobulin domain and Mucin domain 3), VISTA (V-domain Ig suppressor of T cell activation), SIGLEC7 (Sialic acid-binding immunoglobulin-type lectin 7, also called CD328), and SIGLEC9 (Sialic acid-binding immunoglobulin-type lectin 9, also called CD329).

In this regard, the immune suppressor may be an agonist of one or more of the above immune checkpoint molecules. Thus, the immune suppressor may be an immune checkpoint agonist (i.e., to inhibit immune activation). Suitable immune checkpoint agonists, including native ligands and antibodies, are reviewed in Paluch et al Front Immunol, 2018, 9:2306, for example.

Instead of comprising an antigen binding moiety of an antibody, the immune suppressor may comprise one of a receptor-ligand pair, whereby the immune suppressor is capable of binding to the other of the receptor-ligand pair. The target ligand or receptor may be located on an immune cell. For example, the immune suppressor may comprise a ligand of an immune checkpoint molecule described above. In particular, the immune suppressor may comprise a portion (e.g., a soluble extracellular region) of PD-L1 that is capable of binding to PD-1. Such an immune suppressor may engage an immune cell by binding to PD-1 and stimulate PD-1 signalling.

Alternatively the immune suppressor may comprise an agonist antibody that binds to, and may stimulate signalling of, an immune checkpoint molecule. For example, the immune suppressor may be, or comprise, a PD-1 agonist antibody (e.g., single domain antibody). Such PD-1 agonists preferably do not compete with PD-L1 for binding to PD-1. The PD-1 agonist may a full-length antibody or fragment thereof, such as a scFv antibody or a Fab fragment, or a single domain antibody. Examples of such antibodies are provided in WO2011110621 and WO2010029434 and WO2018024237. Thus the antigen of the immune suppressor may be PD-1 and the antigen binding moiety of the immune suppressor may be a PD-1 agonist. The antigen binding moiety of the immune suppressor may comprise a single domain antibody, optionally a VHH. For example, the immune suppressor may be a PD-1 agonist VHH.

As described above, the immune suppressor may be a PD-1 agonist. As used herein, the term “PD-1 agonist” refers to any molecule that is capable of binding to PD-1 and activating PD-1 signalling, including e.g., the PD-1 ligand, PD-L1, and PD-1 agonist antibodies. Activation of the PD-1 pathway down-regulates immune activity, promoting peripheral immune tolerance and preventing autoimmunity (Keir et al., Annu Rev Immunol, 26:677-704, 2008; Okazaki et al., Int Immunol 19:813-824, 2007).

### Half-Life Extending Domains

A “half-life extending domain”, as used herein, refers to a protein domain for extending the half-life of the multi-domain binding protein, relative to a multi-domain binding protein lacking the half-life extending domain. The half-life extending domain comprises a first IgG Fc region (FC1) and a second IgG Fc region (FC2), wherein the FC1 region and FC2 region dimerise to form an Fc domain. As used herein, the term “Fc region” is used to refer to a region of a single polypeptide chain comprising at least a CH2 domain and a CH3 domain sequence, whereas the term “Fc domain” refers to a dimer of two Fc regions (i.e., FC1 and FC2).

The term “half-life” as used herein means a pharmacokinetic property of a binding molecule that is a measure of the mean survival time of binding molecules following their administration. Binding molecule half-life can be expressed as the time required to eliminate 50 percent of a known quantity of a binding molecule from the patient's body (or other mammal) or a specific compartment thereof, for example, as measured in serum, i.e., circulating half-life, or in other tissues.

An increase in half-life allows for the reduction in amount of drug given to a patient as well as reducing the frequency of administration. An increase in half-life can be beneficial, for example, for treatment of cancer, infectious disease or an autoimmune disease or condition. Binding molecules with increased half-lives may also be generated by modifying amino acid residues identified as involved in the interaction between the Fc and the FcRn receptor. Binding molecules comprising Fc regions that comprise one or more modifications which promote binding to FcRn may have an increased half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a binding molecule comprising a native Fc region. Binding molecules comprising Fc regions that comprise one or more modifications which promote binding to FcRn may have an increased half-life of about 2 fold, about 3 fold, about 4 fold, about 5 fold, about 10 fold, about 20 fold, about 50 fold or more, or is between 2 fold and 10 fold, or between 5 fold and 25 fold, or between 15 fold and 50 fold, as compared to binding molecules comprising a native Fc region.

WO 2020/157211 describes an approach for extending the half-life of a TCR-anti-CD3 fusion protein by fusing it to an IgG Fc domain. The present inventors have surprisingly found that the multi-domain binding molecules of the invention retain the extended half-life provided by the Fc domain in the format disclosed in WO 2020/157211, but, in addition, have significantly higher potency.

The immunoglobulin Fc domain may be any antibody Fc domain. The Fc domain is the tail region of an antibody that interacts with cell surface Fc receptors and some proteins of the complement system. The Fc domain comprises two polypeptide chains (i.e., two Fc “regions”) both having two or three heavy chain constant domains (termed CH2, CH3 and CH4), and optionally a hinge region. The two Fc region chains may be linked by one or more disulphide bonds within the hinge region. Fc domains from immunoglobulin subclasses IgG1, IgG2 and IgG4 bind to and undergo FcRn mediated recycling, affording a long circulatory half-life (3-4 weeks), thus extending the half-life of the multi-domain binding molecule of the invention. The interaction of IgG with FcRn has been localized in the Fc region covering parts of the CH2 and CH3 domains. Preferred immunoglobulin Fc domains for use in the present invention include, but are not limited to Fc domains from IgG1 or IgG4. For example, the Fc domain may be an IgG1 Fc domain, i.e., the FC1 and FC2 regions may be IgG1 Fc regions. The Fc domain may be derived from human sequences.

The FC1 region may comprise, or consist of, an amino acid sequence that is at least 80% identical to the sequence of SEQ ID NO: 42 and the FC2 region may comprise, or consist of, an amino acid sequence that is at least 80% identical to the sequence of SEQ ID NO: 43. The FC1 region may comprise, or consist of, an amino acid sequence that is at least 90%, at least 95%, or at least 98% identical to the sequence of SEQ ID NO: 42 and the FC2 region may comprise, or consist of, an amino acid sequence that is at least 90%, at least 95%, or at least 98% identical to the sequence of SEQ ID NO: 43. Preferably, the FC1 region comprises, or consists of, the amino acid sequence provided in SEQ ID NO: 42 and the FC2 region comprises, or consists of, the amino acid sequence provided in SEQ ID NO: 43. As the skilled person would appreciate, the sequences provided above for FC1 and FC2 are suitable vice versa. For example, the FC1 region may comprise, or consist of, the amino acid sequence provided in SEQ ID NO: 43 and the FC2 region may comprise, or consist of, the amino acid sequence provided in SEQ ID NO: 42.

The Fc regions may comprise mutations relative to a wild-type or unmodified Fc sequence. Mutations include substitutions, insertions and deletions. Such mutations may be made for the purpose of introducing desirable therapeutic properties. For example, to facilitate hetero-dimerisation, knobs into holes (KiH) mutations maybe engineered into the CH3 domain. Thus, the half-life extending domain may comprise one or more amino acid substitutions which facilitate dimerisation of the FC1 region and the FC2 region. Such substitutions include “Knob-in-hole” substitutions. In this case, one chain (i.e. one of the FC1 or FC2 regions) is engineered to contain a bulky protruding residue (i.e. the knob), such as Y, and the other chain (i.e., the other of the FC1 and FC2 regions) is engineered to contain a complementary pocket (i.e. the hole). For example, a knob may be constructed by replacing a small amino acid side chain with a larger side chain. A hole may be constructed by replacing a large amino acid side chain with a smaller side chain. Without wishing to be bound to theory, this is thought to stabilize a hetero-dimer of the FC1 and FC2 regions by favouring formation of the hetero-dimer over other species, for example homomultimers of FC1 and FC2, thereby enhancing the stability and manufacturability of the multi-domain binding molecule of the invention.

Suitable positions and substitutions for KiH mutations, and other mutations for facilitating dimerisation of Fc regions, are known in the art and include those described in Merchant et al., Nat Biotechnol 16:677 (1998) and Ridgway et al., Prot Engineering 9:617 (1996) and Atwell et al. J Mol Biol 270,1 (1997): 26-35. For example, the substitutions forming corresponding knobs and holes in two Fc regions may correspond to one or more pairs provided in the following table:

| CH3 of one of the | CH3 of the other of |
|---|---|
| FC1 and FC2 regions | the FC1 and FC2 regions |
| T366Y | Y407T |
| T366W | Y407A |
| T366W | T366S:L368A:Y407V |
| F405A | T394W |
| Y407T | T366Y |
| T366Y:F405A | T394W:Y407T |
| T366W:F405W | T394S:Y407A |
| F405W:Y407A | T366W:T394S |
| F405W | T394S |

The substitutions in the table above are denoted by the original residue, followed by the position using the EU numbering system, and then the import residue (all residues are given in single-letter amino acid code). Multiple substitutions are separated by a colon.

The FC1 and FC2 regions may comprise one or more substitutions in the table above. For example:
(i) one of the FC1 region and the FC2 region may comprise one or more amino acid substitutions selected from the group consisting of T366S, L368A, T394S, F405A, Y407A, Y407T and Y407V, according to the EU numbering scheme; and
(ii) the other of the FC1 region and the FC2 region may comprise one or more amino acid substitutions selected from the group consisting of T366W, T366Y, T366W, T394W and F405W according to the EU numbering scheme. The substitutions in (i) and (ii) are hole-forming and knob-forming substitutions respectively. The FC1 region may comprise one or more of the substitutions in (i) and the FC2 region may comprise one or more of the substitutions in (ii).

### For Example:

(i) one of the FC1 region and the FC2 region may comprise one or more amino acid substitutions selected from the group consisting of T366S, L368A, and Y407V, according to the EU numbering scheme; and
(ii) the other of the FC1 region and the FC2 region may comprise a T366W amino acid substitution, according to the EU numbering scheme. The FC1 region may comprise one or more of the substitutions in (i) and the FC2 region may comprise the substitution in (ii).

Preferably, (i) one of the FC1 region and the FC2 region comprises T366S, L368A, and Y407V amino acid substitutions, according to the EU numbering scheme; and (ii) the other of the FC1 region and the FC2 region comprises a T366W amino acid substitution, according to the EU numbering scheme. For example, the FC1 region may comprise T366S, L368A, and Y407V amino acid substitutions, according to the EU numbering scheme; and the FC2 region may comprise a T366W amino acid substitution, according to the EU numbering scheme. The Fc domain may also comprise one or more mutations that attenuate an effector function of the Fc domain. Exemplary effector functions include, without limitation, complement-dependent cytotoxicity (CDC) and/or antibody-dependent cellular cytotoxicity (ADCC). The modification to attenuate effector function may be a modification that alters the glycosylation pattern of the Fc domain, e.g., a modification that results in an aglycosylated Fc domain. Alternatively, the modification to attenuate effector function may be a modification that does not alter the glycosylation pattern of the Fc domain. The modification to attenuate effector function may reduce or eliminate binding to human effector cells, binding to one or more Fc receptors, and/or binding to cells expressing an Fc receptor. For example, the half-life extending domain may comprise one or more amino acid substitutions selected from the group consisting of S228P, E233P, L234A, L235A, L235E, L235P, G236R, G237A, P238S, F241A, V264A D265A, H268A, D270A, N297A, N297G, N297Q, E318A, K322A, L328R, P329G, P329A, A330S, A330L, P331A and P331S, according to the EU numbering scheme. Particular modifications include a N297G or N297A substitution in the Fc region of human IgG1 (EU numbering). Other suitable modifications include L234A, L235A and P329G substitutions in the Fc region of human IgG1 (EU numbering), that result in attenuated effector function. The Fc regions in the multi-domain binding molecule of the invention may comprise a substitution at residue N297, numbering according to EU index. For example, the substitution may be an N297G or N297A substitution. Other suitable mutations (e.g., at residue N297) are known to those skilled in the art.

Fc variants having reduced effector function refers to Fc variants that reduce effector function (e.g., CDC, ADCC, and/or binding to FcR, etc. activities) by at least 10%, 20%, 30%, 40%, 50%, 60%, 70%, 80%, 90%, 95%, 97%, 98%, 99% or more as compared to the effector function achieved by a wild-type Fc region (e.g., an Fc region not having a mutation to reduce effector function, although it may have other mutations). The Fc variants having reduced effector function may be Fc variants that eliminate all detectable effector function as compared to a wild-type Fc region. Assays for measuring effector function are known in the art and described below.

In vitro and/or in vivo cytotoxicity assays can be conducted to confirm the reduction/depletion of CDC and/or ADCC activities. For example, Fc receptor (FcR) binding assays can be conducted to ensure that the Fc region or fusion protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Substitutions may be introduced into the FC1 and FC2 regions that abrogate or reduce binding to Fcy receptors and/or to increase binding to FcRn, and/or prevent Fab arm exchange, and/or remove protease sites. In this regard, the half-life extending domain may also comprise one or more amino acid substitutions which prevent or reduce binding to activating receptors. The half-life extending domain may comprise one or more amino acid substitutions which prevent or reduce binding to FcγR. For example, the FC1 region and/or the FC2 region may comprise a N297G amino acid substitution, according to the EU numbering scheme. Both the FC1 region and the FC2 region may comprise the N297G amino acid substitution.

The half-life extending domain may comprise one or more amino acid substitutions compared to the unmodified half-life extending domain, wherein the one or more amino acid substitutions promote binding of the Fc domain to FcRn. Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody substitutions which improved or diminished binding to FcRs. See also, e.g., Shields et al., J. Biol. Chem. 9 (2): 6591-6604 (2001). In particular, Mackness et al., MAbs. 11:1276-1288 (2019) describes suitable amino acid substitutions in antibody Fc regions for enhancing binding to FcRn.

Modification(s) (e.g., amino acid substitutions, amino acid insertions, or amino acid deletions) in a Fc region which promote binding of the Fc domain to FcRn may be at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, a Fc region may comprise a non-naturally occurring amino acid residue at additional and/or alternative positions known in the art.

More specifically, a Fc region can comprise at least one substitution selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 262I, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 265I, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 299I, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, a Fc region may comprise additional and/or alternative non-naturally occurring amino acid residues known in the art.

The modification(s) (e.g., amino acid substitutions, amino acid insertions, or amino acid deletions) in a Fc region which promote binding to FcRn may be at one or more positions selected from the group consisting of 234, 235 and 331, as numbered by the EU index as set forth in Kabat. For example, a Fc region may comprise at least one substitution selected from the group consisting of 234F, 235F, 235Y, and 331S, as numbered by the EU index as set forth in Kabat.

The modification(s) (e.g., amino acid substitutions, amino acid insertions, or amino acid deletions) in a Fc region which promote binding to FcRn may be at one or more positions selected from the group consisting of 239, 330 and 332, as numbered by the EU index as set forth in Kabat. For example, a Fc region may comprise at least one substitution selected from the group consisting of 239D, 330L and 332E, as numbered by the EU index as set forth in Kabat.

The modification(s) (e.g., amino acid substitutions, amino acid insertions, or amino acid deletions) in a Fc region which promote binding to FcRn may be at one or more positions selected from the group consisting of 252, 254, and 256, as numbered by the EU index as set forth in Kabat. For example, a Fc region may comprise at least one substitution selected from the group consisting of 252Y, 254T and 256E, as numbered by the EU index as set forth in Kabat, as described in U.S. Pat. No. 7,083,784, the contents of which are herein incorporated by reference in its entirety. A Fc region may comprise all of the following substitutions: 252Y, 254T and 256E, as numbered by the EU index as set forth in Kabat.

The substitutions which promote binding to FcRn listed above are relative to a corresponding wild-type Fc region (e.g., a human IgG1 or IgG4 Fc region) and may be present in one of or preferably both of the FC1 and FC2 portions of the Fc domain. In other words, the substitutions refer to amino acids that are not normally present in a corresponding wild-type Fc region, for example a human IgG1 or IgG4 Fc region. In this regard, a “substitution”, as used herein, refers to the presence of one of the listed amino acids in a polypeptide and does not necessarily require replacing one amino acid with another. In one embodiment, the FC1 and/or FC2 region comprise 252Y, 254T and 256E amino acid substitutions, numbered according to the EU numbering scheme.

Additionally or alternatively, mutations may be made for manufacturing reasons, for example to remove or replace amino acids that may be subject to post-translational modifications such as glycosylation, as described herein. The immunoglobulin Fc may be fused to the other domains (i.e., VC1 or VC2) in the molecule of the invention via a linker, and/or a hinge sequence as described herein. Alternatively no linker may be used.

The FC1 region may comprise, or consist of, an amino acid sequence that is at least 80% identical to the sequence of SEQ ID NO: 57 and the FC2 region may comprise, or consist of, an amino acid sequence that is at least 80% identical to the sequence of SEQ ID NO: 58. The FC1 region may comprise, or consist of, an amino acid sequence that is at least 90%, at least 95%, or at least 98% identical to the sequence of SEQ ID NO: 57 and the FC2 region may comprise, or consist of, an amino acid sequence that is at least 90%, at least 95%, or at least 98% identical to the sequence of SEQ ID NO: 58. The FC1 region may comprise, or consist of, the amino acid sequence provided in SEQ ID NO: 57 and the FC2 region may comprise, or consist of, the amino acid sequence provided in SEQ ID NO: 58. As the skilled person would appreciate, the sequences provided above for FC1 and FC2 are suitable vice versa. For example, the FC1 region may comprise, or consist of, the amino acid sequence provided in SEQ ID NO: 57 and the FC2 region may comprise, or consist of, the amino acid sequence provided in SEQ ID NO: 58.

The two Fc regions in the molecule of the invention may comprise CH2 and CH3 constant domains and all or part of a hinge sequence. The hinge sequence may correspond substantially or partially to a hinge region from IgG1, IgG2, IgG3 or IgG4. The hinge sequence may be an IgG1 hinge sequence, such as the amino acid sequence provided in SEQ ID NO: 44. The hinge may comprise all or part of a core hinge domain and all or part of a lower hinge region.

Suitable half-life extended formats of multi-domain binding molecules of the invention are also described in an application filed herewith, entitled “Multi-Domain Binding Molecules,” which claims priority benefit of U.S. Prov. Appl. No. 63/371,863, filed Aug. 18, 2022, the contents of which are herein incorporated by reference.

### Format and Linkers

As used herein, the term “format” refers to the position and orientation of each domain (and each region in each domain), and the number of polypeptide chains, in the multi-domain binding molecule of the invention. A schematic diagram of the format of an exemplary multi-domain binding molecule is provided in FIG. 1. The immune cell engaging domain in the exemplary binding molecule shown in FIG. 1 is a T cell engaging immune effector domain comprising a VH (TCE-VH) and a VL (TCE-VL).

Other types of immune cell engaging domains (e.g., a single domain antibody, VHH etc) are also suitable, as described herein above. The pMHC binding domain and the immune cell engaging domain of such molecules are capable of binding to a pMHC complex and an immune cell, respectively. In this regard, the pMHC binding domain and the immune cell engaging domain may be capable of simultaneously binding to a pMHC complex and an immune cell, respectively.

In the format of the multi-domain binding molecule of the invention, the immune cell engaging domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of FC2. Each region is linked covalently in a single polypeptide chain. The format can be represented as: N-ICE-VC1-FC1-VC2-FC2-C. The inventors have identified that molecules in this format have the highest activity (i.e., potency and selectivity) and production yield of the more than 35 different formats tested.

Where the immune cell engaging domain is a T cell engaging immune effector domain comprising a VH and VL, the format can be represented as: N-(TCEVL-TCEVH or TCEVH-TCEVL)-VC1-FC1-VC2-FC2-C. The inventors have identified that molecules in this format have the highest activity (i.e., potency and selectivity) and production yield of the more than 35 different formats tested.

The multi-domain binding molecule of the invention is in a single-chain format. In this context, “single-chain” is used to describe a multi-domain binding molecule that is expressed as a single polypeptide chain which contains the pMHC binding domain, the immune cell engaging domain and the half-life extending domain.

Preferably, VC1 comprises a TCRβ variable and constant region, VC2 comprises a TCRα variable and constant region, the immune cell engaging domain is an anti-CD3 scFv and the Fc domain is an IgG1 Fc domain.

Two or more of the ICE, TCE-VH, TCE-VL, VC1, VC2, FC1 and/or FC2 regions may be linked to each other via linkers and/or IgG hinge sequences. Linker sequences may be flexible, in that they are made up primarily of amino acids such as glycine, alanine and serine, which do not have bulky side chains likely to restrict flexibility. Such linkers include “glycine-serine” linkers, which refer to linkers that comprise only, or predominantly, glycine and serine residues for example (GGGGS) n. Alternatively, linkers with greater rigidity may be desirable. Examples of more rigid linkers include alpha helix-forming linkers with the sequence of (EAAAK) n. Usable or optimum lengths of linker sequences may be easily determined. Often the linker sequence will be less than about 15, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules are known in the art and include those described in WO2010/133828 and Chen et al Adv Drug Deliv Rev. 2013; 65 (10): 1357-1369. For example, the linker or linkers present in the multi-domain binding protein of the invention may have a sequence selected from the group of GGGGS (SEQ ID NO: 18), GGGSG (SEQ ID NO: 20), GGSGG (SEQ ID NO: 21), GSGGG (SEQ ID NO: 22), GSGGGP (SEQ ID NO: 23), GGEPS (SEQ ID NO: 24), GGEGGGP (SEQ ID NO: 25), GGEGGGSEGGGS (SEQ ID NO: 26), GGGSGGGG (SEQ ID NO: 47), GGGGSGGGGSGGGGSGGGGSGGGS (SEQ ID NO: 39), GGGGGGGGSGGGGS (SEQ ID NO: 49), EAAAK (SEQ ID NO: 50) and EAAAKEAAAKEAAAK (SEQ ID NO: 51).

Suitable IgG hinge sequences are known in the art and include the exemplary IgG1 hinge sequence provided in SEQ ID NO: 44. Other suitable IgG hinge sequences include a truncated IgG1 hinge sequence provided in SEQ ID NO: 52 and the IgG4 hinge provided in SEQ ID NO: 53.

The ICE domain may be linked via its C-terminus to the N-terminus of VC1. In this regard, the multi-domain binding molecule of the invention may have the following format: N-ICE-VC1-FC1-VC2-FC2-C. Where the immune cell engaging domain is a T cell engaging immune effector domain comprising a VH and VL, the TCE-VL region may be linked via its C terminus to the N terminus of the TCE-VH region and the TCE-VH region may be linked via its C terminus to the N terminus of VC1. In this regard, the multi-domain binding molecule of the invention may have the following format: N-TCEVL-TCEVH-VC1-FC1-VC2-FC2-C.

VC1 may comprise a TCRβ variable and constant region and VC2 may comprise a TCRα variable and constant region. Thus, VC1 and VC2 may dimerise to form a soluble TCR. In this regard, preferably, the multi-domain binding molecule of the invention has the following format: N-ICE-TCRβ-FC1-TCRα-FC2-C or N-TCEVL-TCEVH-TCRβ-FC1-TCRα-FC2-C(where “TCRβ” refers to the TCRβ variable and constant region and “TCRα” refers to the TCRα variable and constant region.

If present, the TCE-VL region may be linked to the TCE-VH region via a sequence comprising a glycine-serine linker. Preferably, the sequence linking the TCE-VL region to the TCE-VH region is the amino acid sequence provided in SEQ ID NO: 39.

The ICE domain or TCE-VH region may be linked to VC1 via a sequence comprising, or consisting of, a glycine-serine linker. Preferably, the sequence linking the ICE domain or TCE-VH region to VC1 is the amino acid sequence provided in SEQ ID NO: 18.

VC1 may be linked to the FC1 region via a sequence comprising an IgG hinge sequence and/or VC2 may be linked to the FC2 region via a sequence comprising an IgG hinge sequence. The IgG hinge sequence may be at least 80% identical to SEQ ID NO: 44. Preferably, the IgG hinge sequence is at least 90%, at least 95%, at least 98% or is 100% identical to SEQ ID NO: 44.

The sequence linking VC1 to the FC1 region may further comprise a glycine-serine linker and/or the sequence linking VC2 to the FC2 region may further comprise a glycine-serine linker. Preferably, the glycine-serine linker has the sequence provided in SEQ ID NO: 47. Preferably, these sequences are in the following formats, N-terminal to C-terminal: VC1-GS linker-IgG hinge-FC1 and VC2-GS linker-IgG hinge-FC2.

The FC1 region may be linked to VC2 via a sequence comprising a glycine-serine linker. Preferably, the glycine-serine linker linking the FC1 region VC2 region has the sequence provided in SEQ ID NO: 47.

The multi-domain binding molecule of the invention is a single polypeptide chain (see FIG. 1). The multi-domain binding molecule may be soluble and/or recombinant and/or isolated. Complete amino acid sequences of two exemplary multi-domain binding molecules are provided in SEQ ID NO: 45 and SEQ ID NO: 46.

The multi-domain binding molecule may have an amino acid sequence that is at least 80% identical to the sequence of SEQ ID NO: 45. The multi-domain binding molecule may have an amino acid sequence that is at least 90%, at least 95%, or at least 98% identical to the sequence of SEQ ID NO: 45. Preferably, the multi-domain binding molecule comprises, or consists of, the amino acid sequence provided in SEQ ID NO: 45.

The multi-domain binding molecule may have an amino acid sequence that is at least 80% identical to the sequence of SEQ ID NO: 46. The multi-domain binding molecule may have an amino acid sequence that is at least 90%, at least 95%, or at least 98% identical to the sequence of SEQ ID NO: 46. The multi-domain binding molecule may comprise, or consist of, the amino acid sequence provided in SEQ ID NO: 46.

Optionally, the multi-domain binding molecule sequences above may be further fused to one or more other polypeptide sequences.

The sequences above relate to multi-domain binding molecules comprising TCR chains that bind to a SLLQHLIGL (SEQ ID NO: 1) HLA-A*02 complex. A person skilled in the art could adapt these sequences to another target by replacing the TCR chains in SEQ ID NO: 45 and SEQ ID NO: 46 with sequences of a different TCR of interest. Similarly, the person skilled in art could replace the anti-CD3 scFv sequence (i.e., the T Cell engaging immune effector domain) in SEQ ID NO: 45 or SEQ ID NO: 46 with another T Cell engaging immune effector domain, e.g., a different anti-CD3 scFv sequence.

Preferably:
a) VC1 comprises a TCRβ variable and constant region,
b) VC2 comprises a TCRα variable and constant region,
c) the immune cell engaging domain is a T cell engaging immune effector domain which is an anti-CD3 scFv,
d) FC1 has the amino acid sequence provided in SEQ ID NO: 42, or an amino acid sequence at least 90%, or at least 95%, or at least 98% identical thereto, and
e) FC2 has the amino acid sequence provided in SEQ ID NO: 43, or an amino acid sequence at least 90%, at least 95%, or at least 98% identical thereto.

The multi-domain binding molecule preferably comprises the following amino acid sequences, in the following order from N-terminus to C-terminus:
a) an amino acid sequence of an anti-CD3 scFv (TCE-VL and TCE-VH), optionally followed by a linker sequence provided in SEQ ID NO: 18;
b) an amino acid sequence of a TCRβ variable and constant region (VC1);
c) a linker sequence provided in SEQ ID NO: 47 followed by an IgG hinge sequence provided in SEQ ID NO: 44;
d) an Fc region having the sequence provided in SEQ ID NO: 42 (FC1);
e) a linker sequence provided in SEQ ID NO: 47;
f) an amino acid sequence of a TCRα variable and constant region (VC2);
g) a linker sequence provided in SEQ ID NO: 47 followed by an IgG hinge sequence provided in SEQ ID NO: 44; and
h) an Fc region having the sequence provided in SEQ ID NO: 43 (FC2).

The TCRβ constant region may have the amino acid sequence provided in SEQ ID NO: 19 and/or the TCRα constant region may have the amino acid sequence provided in SEQ ID NO: 15. The multi-domain binding molecule may comprise no amino acid sequences other than the sequences in a) to h) above.

The anti-CD3 scFv may comprise, or consist of, the amino acid sequence provided in SEQ ID NO: 17 or the amino acid sequence provided in SEQ ID NO: 40.

### Amino Acid Sequences

Within the scope of the invention are phenotypically silent variants of any molecule disclosed herein. As used herein the term “phenotypically silent variants” is understood to refer to a variant which incorporates one or more further amino acid changes, including substitutions, insertions and deletions, in addition to those set out above, and which variant has a similar phenotype to the corresponding molecule without said change(s). For the purposes of this application, phenotype comprises binding affinity (K_{D} and/or binding half-life) and specificity. The phenotype for a soluble multi-domain binding molecule may include potency of immune activation and purification yield, in addition to binding affinity and specificity.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a molecule comprising any of the amino acid sequences described above but with one or more conservative substitutions and or one or more tolerated substitutions in the sequence, such that the amino acid sequence of the molecule, or any domain or region thereof, has at least 90% identity, such as 90%, 91%, 92%, 93%, 94%, 95%, 96%, 97%, 98%, 99% or 100% identity, to the sequences disclosed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size=3, Expect Threshold=10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the molecule, for example a TCR portion. The sequences provided at the C-terminus and/or N-terminus thereof may be truncated or extended by 1, 2, 3, 4 or 5 residues. All such variants are encompassed by the present invention.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning-A Laboratory Manual (3rd Ed.) CSHL Press. Further information on ligation independent cloning (LIC) procedures can be found in Rashtchian, (1995) Curr Opin Biotechnol 6 (1): 30-6. The protein sequences provided herein may be obtained from recombinant expression, solid state synthesis, or any other appropriate method known in the art.

### Assessing Binding Characteristics and Activity of Multi-Domain Binding Molecules

Methods to determine binding affinity (inversely proportional to the equilibrium constant K_{D}) and binding half-life (expressed as T1/2) are known to those skilled in the art. Binding affinity and binding half-life may be determined using Surface Plasmon Resonance (SPR) or Bio-Layer Interferometry (BLI), for example using a BIAcore instrument or Octet instrument, respectively. It will be appreciated that doubling the affinity results in halving the K_{D}. T1/2 is calculated as In2 divided by the off-rate (K_{off}). Therefore, doubling of T1/2 results in a halving in K_{off}. K_{D} and K_{off} values for TCRs are usually measured for soluble forms of the TCR, i.e. those forms which are truncated to remove cytoplasmic and transmembrane domain residues. To account for variation between independent measurements, and particularly for interactions with dissociation times in excess of 20 hours, the binding affinity and or binding half-life of a given protein may be measured several times, for example 3 or more times, using the same assay protocol, and an average of the results taken. To compare binding data between two samples (i.e. two different proteins and or two preparations of the same protein) it is preferable that measurements are made using the same assay conditions (e.g. temperature). Measurement methods described in relation to TCRs may also be applied to the multi-domain binding molecules described herein.

Certain multi-domain binding molecules of the invention are able to generate a highly potent T cell response in vitro against antigen positive cells, in particular those cells presenting low levels of antigen typical of cancer cells (i.e. in the order of 5-100, for example 50, antigens per cell (Bossi et al., (2013) Oncoimmunol. 1; 2 (11):e26840; Purbhoo et al., (2006). J Immunol 176(12): 7308-7316). Such TCRs may be suitable for incorporation into the multi-domain binding molecules described herein. The T cell response that is measured may be the release of T cell activation markers such as Interferon γ or Granzyme B, or target cell killing, or other measure of T cell activation, such as T cell proliferation. A highly potent response may be one with an EC₅₀ value in the nM-pM range, for example 500 nM or lower, preferably 1 nM or lower, or 500 μM or lower.

Alternatively, certain binding molecules of the invention may generate a highly potent anti-inflammatory response, such as CD8+ cell killing and/or CD4+ inflammation inhibition. Such binding molecules may be in soluble form and may comprise an immune cell engaging domain which is an immune suppressor such as a PD-1 agonist or an interleukin or cytokine such as IL-2, IL-4, IL-10 or IL-13. The anti-inflammatory response that is measured may be CD8+ cell killing and/or CD4+ inflammation inhibition, and or inhibition of CD8+ T cell signalling pathways. Suitable methods for assessing an anti-inflammatory response will be known in the art and include Jurkat NFAT cell reporter assays. Preferably a highly potent response is one with IC50 value in the pM range, i.e. 1000 pM or lower. Preferably the maximum inhibition obtained in reporter assays is greater than 50%, for example 80% or more.

Molecules encompassed by the present invention may have an improved half-life. Methods for determining whether a protein has an improved half-life will be apparent to the skilled person. For example, the ability of a protein to bind to a neonatal Fc receptor (FcRn) is assessed. In this regard, increased binding affinity for FcRn increases the serum half-life of the protein (see for example, Kim et al. Eur J Immunol., 24:2429, 1994).

The half-life of a protein of the disclosure can also be measured by pharmacokinetic studies, e.g., according to the method described by Kim et al. Eur J of Immunol 24:542, 1994. According to this method radiolabeled protein is injected intravenously into mice and its plasma concentration is periodically measured as a function of time, for example at 3 minutes to 72 hours after the injection.

Alternatively, unlabelled protein of the disclosure can be injected and its plasma concentration periodically measured using an ELISA. The clearance curve thus obtained should be biphasic, that is, an alpha phase and beta phase. For the determination of the in vivo half-life of the protein, the clearance rate in beta-phase is calculated and compared with that of the wild type or unmodified protein.

### Nucleic Acids, Vectors and Host Cells

The present invention provides a nucleic acid encoding a multi-domain binding molecule of the invention. The nucleic acid may be cDNA. The nucleic acid may be mRNA. The nucleic acid may be non-naturally occurring and/or purified and/or engineered. The nucleic acid sequence may be codon optimised, in accordance with the expression system utilised. As is known to those skilled in the art, expression systems may include bacterial cells such as E. coli, or yeast cells, or mammalian cells, or insect cells, or they may be cell free expression systems.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a specific binding molecule of the invention forms an aspect of the present invention, as does a method of production of the specific binding molecule comprising expression from a nucleic acid encoding a specific binding molecule of the invention. Expression may conveniently be achieved by culturing recombinant host cells containing the nucleic acid under appropriate conditions. Following production by expression, a specific binding molecule may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, Hela cells, baby hamster kidney cells, NSO mouse melanoma cells and many others. A common, preferred bacterial host is E. coli. The expression of antibodies and antibody fragments in prokaryotic cells such as E. coli is well established in the art. For a review, see for example Pluckthun, Bio/Technology 9:545-551 (1991). Expression in eukaryotic cells in culture is also available to those skilled in the art as an option for production of a specific binding molecule, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be any suitable vectors known in the art, including plasmids or viral vectors (e.g. phage, or phagemid), as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The present invention also provides a host cell containing a nucleic acid as disclosed herein. Further, the invention provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

Suitable host cells for cloning or expression of polynucleotides and/or vectors of the present invention are known in the art. Suitable host cells for the expression of (glycosylated) proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F.L. et al., J. Gen Virol. 36 (1977) 59-74); baby hamster kidney cells (BHK); mouse Sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NSO and Sp2/0. For a review of certain mammalian host cell lines suitable for protein production, see, e.g., Yazaki, P. and Wu, A. M., Methods in Molecular Biology, Vol. 248, Lo, B. K. C. (ed.), Humana Press, Totowa, NJ (2004), pp. 255-268. The host cell may be eukaryotic, e.g., a Chinese Hamster Ovary (CHO) cell or lymphoid cell (e.g., Y0, NSO, Sp20 cell).

The nucleic acid of the invention may be integrated into the genome (e.g. chromosome) of the host cell. Integration may be promoted by inclusion of sequences which promote recombination with the genome, in accordance with standard techniques.

### Methods of Making Multi-Domain Binding Molecules

Further provided herein are methods for making the multi-domain binding molecule described herein. The methods comprise maintaining the host cell of the invention under optimal conditions for expression of the nucleic acid or expression vector of the invention and isolating the multi-domain binding molecule.

Methods of producing recombinant proteins are well known in the art. Nucleic acids encoding the protein can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary mammalian cells used for expressing a protein are CHO cells, myeloma cells or HEK cells. Molecular cloning techniques to achieve these ends are known in the art and described, for example in Ausubel et al. (editors), Current Protocols in Molecular Biology, Greene Pub. Associates and Wiley-Interscience (1988, including all updates until present) or Sambrook et al. Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

The nucleic acid may be inserted operably linked to a promoter in an expression construct or expression vector for further cloning (amplification of the DNA) or for expression in a cell-free system or in cells. As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid. As used herein, the term “operably linked to” means positioning a promoter relative to a nucleic acid such that expression of the nucleic acid is controlled by the promoter.

Many vectors for expression in cells are commercially available. The vector components generally include, but are not limited to, one or more of the following: a signal sequence, a sequence encoding a protein (e.g., derived from the information provided herein), an enhancer element, a promoter, and a transcription termination sequence. The skilled person will be aware of suitable sequences for expression of a protein. Exemplary signal sequences include prokaryotic secretion signals (e.g., pe1B, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-a promoter (EF1), small nuclear RNA promoters (Ula and Ulb), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or an active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GALA promoter, the CUP1 promoter, the PH05 promoter, the nmt promoter, the RPR1 promoter, or the TEF1 promoter.

The host cells used to produce the protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

Methods for isolating a protein are known in the art. Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing.

These methods are known in the art and described, for example in WO99/57134 or Ed Harlow and David Lane (editors) Antibodies: A Laboratory Manual, Cold Spring Harbor Laboratory, (1988). The skilled person will also be aware that a protein can be modified to include a tag to facilitate purification or detection, e.g., a poly-histidine tag, a hexa-histidine tag, an influenza virus hemagglutinin (HA) tag, a Simian Virus 5 (V5) tag, a LLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Molecules of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or greater than 5%, or higher yield. High yield means greater than 1 mg/ml, or greater than 3 mg/ml, or greater than 5 mg/ml, or higher yield.

### Pharmaceutical Compositions and Medical Methods

For administration to patients, the molecules of the invention, nucleic acids, expression vectors or cells of the invention may be provided as part of a pharmaceutical composition together with one or more pharmaceutically acceptable carriers or excipients. This pharmaceutical composition may be in any suitable form, (e.g. depending upon the desired method of administering it to a patient). It may be provided in unit dosage form, and will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions. Methods for preparing a protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions will commonly comprise a solution of the multi-domain binding molecule of the invention (or the nucleic acid, cell, or vector of the invention) dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of molecules of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Molecules of the invention may have an ideal safety profile for use as therapeutic agents. An ideal safety profile means that in addition to demonstrating good specificity, the molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Dosages of the molecules of the present invention can vary between wide limits, depending upon the disease or disorder to be treated, the age and condition of the individual to be treated, etc. A physician will ultimately determine appropriate dosages to be used.

Multi-domain binding molecules, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

The multi-domain binding molecule of the invention may be further associated with a therapeutic agent. Therapeutic agents which may be associated with the molecules of the invention include immune-modulators and effectors, radioactive compounds, enzymes (perforin for example) or chemotherapeutic agents (cis-platin for example). To ensure that toxic effects are exercised in the desired location the toxin could be inside a liposome linked to the multi-domain binding molecule described herein so that the compound is released slowly. This will prevent damaging effects during the transport in the body and ensure that the toxin has maximum effect after binding of the multi-domain binding molecule described herein to the relevant antigen presenting cells.

Examples of suitable therapeutic agents include, but are not limited to:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E, vincristine and doxorubicin;
peptide cytotoxins, i.e. proteins or fragments thereof with the ability to kill mammalian cells. For example, ricin, diphtheria toxin, pseudomonas bacterial exotoxin A, Dnase and Rnase;
radio-nuclides, i.e. unstable isotopes of elements which decay with the concurrent emission of one or more of a or B particles, or γ rays. For example, iodine 131, rhenium 186, indium 111, yttrium 90, bismuth 210 and 213, actinium 225 and astatine 213; chelating agents may be used to facilitate the association of these radio-nuclides to the multi-domain binding molecule;
immuno-stimulants, i.e. immune effector molecules which stimulate immune response. For example, cytokines such as IL-2 and IFN-γ,
superantigens and mutants thereof;
TCR-HLA fusions, e.g. fusion to a peptide-HLA complex, wherein said peptide is derived from a common human pathogen, such as Epstein Barr Virus (EBV);
chemokines such as IL-8, platelet factor 4, melanoma growth stimulatory protein, etc;
antibodies or fragments thereof, including anti-T cell or NK cell determinant antibodies (e.g. anti-CD3, anti-CD28 or anti-CD16);
antibodies or fragments thereof that bind to molecules that locate to the immune synapse;
alternative protein scaffolds with antibody like binding characteristics;
complement activators;
xenogeneic protein domains, allogeneic protein domains, viral/bacterial protein domains, viral/bacterial peptides.

The multi-domain binding molecule, nucleic acid, vector, pharmaceutical composition and cell of the invention may be used for treating diseases such as cancer, particularly cancers which are associated with expression of a tumour-associated antigen. For example, the cancer may be associated with expression of GP100, NYESO, MAGEA4, or PRAME as described in WO2011001152, WO2017109496, WO2017175006 and WO2018234319, and, for example, in corresponding U.S. Pat. Nos. 8,519,100, 11,639,374, 11,505,590, and 11,427,624, the contents of each which are herein incorporated by reference.

The cancer to be treated may be a cancer associated with PRAME expression. By “associated with PRAME expression” it is meant that the cancer comprises cancer cells that express PRAME. In this regard, the cancer may be a PRAME-positive cancer. The cancer may be known to be associated with expression of PRAME, and thus PRAME expression may not be assessed. Alternatively, PRAME expression can be assessed using any method known in the art, including, for example, histological methods. However, the invention is not intended to be limited to the treatment of cancers for which PRAME expression can be detected by histological methods. Cancers associated with PRAME expression include, but are not limited to, melanoma, lung cancer, breast cancer, ovarian cancer, endometrial cancer, oesophageal cancer, bladder cancer, head and neck cancer, uterine cancer, Acute myeloid leukemia, chronic myeloid leukemia, and Hodgkin's lymphoma. For example, the cancer associated with PRAME expression may be melanoma. The melanoma may be uveal melanoma or cutaneous melanoma. The lung cancer may be non-small cell lung carcinoma (NSCLC) or small cell lung cancer (SCLC). The breast cancer may be triple-negative breast cancer (TNBC) The bladder cancer may be urothelial carcinoma. The oesophageal cancer may be gastroesophageal junction (GEJ) adenocarcinoma. The ovarian cancer may be epithelial ovarian cancer, such as high grade serous ovarian cancer.

The multi-domain binding molecule, nucleic acid, vector, pharmaceutical composition and cell of the invention may be used for treating an infectious disease. The infectious disease may be caused by a bacterial, viral, fungal or parasitic pathogen. Any infection with a pathogen which results in antigen-presenting cells presenting MHC bound to a peptide from the pathogen may be suitable for treatment with the multi-domain binding molecule of the invention. The multi-domain binding molecule of the invention is particularly well suited to infections where antigen-presenting cells present levels of pathogen peptide that are lower than optimal for the natural immune system to clear the infection without additional treatment. The infectious disease may be a chronic infection. Exemplary infectious diseases include Hepatitis B virus (HBV) infection and human immunodeficiency virus (HIV) infection.

The multi-domain binding molecule of the invention may be used in a method of treating an autoimmune disease, such as type 1 diabetes. Organ-specific immune suppression, rather than systemic immunosuppression, may be a beneficial route for treatment given the potential significant adverse events associated with systemic immunosuppression. In autoimmunity, there is also mounting evidence that PD-1 pathway impairment plays an important role in disease pathogenesis. PD-1, PD-L1 and PD-L2 gene polymorphisms are associated with several autoimmune diseases. Abnormally low PD-L1 expression has been observed in samples from type 1 diabetes and Crohn's disease patients. Activating PD-1 on autoreactive lymphocytes thus may serve as a mechanism to treat autoimmune diseases. Effective therapeutics for the treatment of autoimmune diseases include those having an advantageous risk profile (e.g., a high level of target and tissue specificity) and are capable of being administered with less frequency.

Also provided by the invention are:
a multi-domain binding molecule, nucleic acid, vector, pharmaceutical composition or cell of the invention for use in medicine, preferably for use in a method of treating cancer or a tumour or an infectious disease;
a multi-domain binding molecule, nucleic acid, vector, pharmaceutical composition or cell of the invention for use in medicine, preferably for use in a method of treating cancer or a tumour or an autoimmune disease or an infectious disease;
the use of a multi-domain binding molecule, nucleic acid, vector, pharmaceutical composition or cell of the invention in the manufacture of a medicament for treating cancer or a tumour or an infectious disease;
the use of a multi-domain binding molecule, nucleic acid, vector, pharmaceutical composition or cell of the invention in the manufacture of a medicament for treating cancer or a tumour or an autoimmune disease or infectious disease;
a method of treating cancer or a tumour or an infectious disease in a patient, comprising administering to the patient a multi-domain binding molecule, nucleic acid, vector, pharmaceutical composition or cell of the invention;
a method of treating cancer or a tumour or an autoimmune disease or infectious disease in a patient, comprising administering to the patient a multi-domain binding molecule, nucleic acid, vector, pharmaceutical composition or cell of the invention; and
an injectable formulation for administering to a human subject comprising a multi-domain binding molecule, nucleic acid, vector pharmaceutical composition or cell of the invention.

The method of treatment may further include administering separately, in combination, or sequentially, an additional anti-neoplastic agent. Example of such agents are known in the art and may include immune activating agents and/or T cell modulating agents.

### Kits and Articles of Manufacture

In another aspect, a kit or an article of manufacture containing materials useful for the treatment and/or prevention of the diseases described above is provided.

The kit may comprise (a) a container comprising the molecule, nucleic acid, vector or cell of the invention, optionally in a pharmaceutically acceptable carrier or diluent; and (b) a package insert with instructions for treating a disease (e.g., cancer, immune disease or autoimmune disease) in a subject. The kit may further comprise (c) at least one further therapeutically active compound or drug.

The package insert may be on or associated with the container. Suitable containers include, for example, bottles, vials, syringes, etc. The containers may be formed from a variety of materials such as glass or plastic. The container holds or contains a composition that comprises the molecule, nucleic acid, vector or cell of the invention and may have a sterile access port (for example, the container may be an intravenous solution bag or a vial having a stopper pierceable by a hypodermic injection needle). At least one active agent in the composition is the molecule, nucleic acid, vector or cell of the invention. The label or package insert indicates that the composition is used for treating a subject eligible for treatment, e.g., one having or predisposed to developing a disease described herein, with specific guidance regarding dosing amounts and intervals of the composition and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprising a second medicament, wherein the molecule, nucleic acid, vector or cell of the invention is a first medicament, and which kit further comprises instructions on the package insert for treating the subject with the second medicament, in an effective amount.

Various modifications of the invention in addition to those shown and described herein will become apparent to those skilled in the art from the foregoing description and fall within the scope of the appended claims. Preferred features of each aspect of the invention are as for each of the other aspects mutatis mutandis. The documents referred to herein are incorporated by reference to the fullest extent permitted by law.

### DESCRIPTION OF THE DRAWINGS

FIG. 1 is a schematic diagram of an exemplary multi-domain, single-chain binding molecule of the invention. FIG. 1a shows a representation of the domain arrangement from the N- to C-terminus and FIG. 1b shows a hypothetical representation of the folded structure the molecule.

FIG. 2 shows the results of an ELISpot assay, using IFNγ as a read out of T cell activation. As a comparison, the same TCR was used to construct a multidomain molecule using the previously disclosed format in WO 2020/157211 and tested alongside the single chain format presented in FIG. 1. A schematic representation of each format is positioned in FIG. 2 to indicate the corresponding data points.

FIG. 3 shows graphs of surface plasmon resonance experiments for assessing binding of mol093v9 and mol093v11 to each of pHLA, CD3 and FcRn.

FIG. 4 shows pharmacokinetic properties assessed in Tg32 SCID mice. Mice were dosed by IV bolus at 1 mg/Kg, with serial sampling of blood over a 21 day period. Sample was detected in serum by electrochemiluminescent immunoassay. Graph shows serum concentration over time for 4 individual mice.

FIG. 5 presents graphs showing the results of ELISPot assays in which the T cell activation of mol093v9 and mol093v11 was assessed in vitro.

FIG. 6 presents graphs showing the results of ELISPot assays in which the T cell activation of mol093v9 was compared directly with an alternative molecule targeting the same PRAME peptide, but which does not include the half-life extending Fc domain (WO 2018/234319). FIG. 6 shows that both molecules drive a similarly potent T cell response.

FIG. 7 shows graphs demonstrating real-time killing, as determined using the xCELLigence platform, of antigen positive cells in the presence of mol093v9 and mol093v11.

FIG. 8 presents graphs showing the results of T cell killing assays in which mol093v9 was compared directly with an alternative molecule targeting the same PRAME peptide, but which does not include the half-life extending Fc domain (WO 2018/234319). FIG. 8 shows that mol093v9 demonstrates comparable killing data to the non-HLE version of the molecule (“Mol001”).

FIG. 9 shows data from ELISPOT T cell activation assays obtained with two normal cell lots (cardiac cells (HCM27) and lung epithelial cells (HSAEpiC9)) for one PBMC effector donor. Minical T cell activation against normal cells was observed for concentrations of mol093v9 and mol093v11 up to and including 1.1 nM of fusion molecule.

FIG. 10 presents graphs showing the results of ELISPOT T cell activation assays in which normal cell reactivity for mol093v9 was compared directly with an alternative molecule targeting the same PRAME peptide, but which does not include the half-life extending Fc domain WO 2018/234319. FIG. 10 shows that both molecules show a similar lack of reactivity against normal cells from skin (melanocytes) and kidney (renal proximal tubule).

FIG. 11 is a graph showing T cell activation measured by IFNγ release for TCR anti-CD3 fusion molecule a40b23U28-mol93 in the presence of antigen positive and antigen negative cancer cell lines.

FIG. 12 is a graph showing T cell activation measured by IFNγ release for half-life extended TCR anti-CD3 fusion molecules a40b23U28-mol93 and a40b23U28-mol14 in the presence of antigen positive cells.

### DESCRIPTION OF THE SEQUENCES

SEQ ID NO: 1 HLA-A*02 restricted peptide: SLLQHLIGL

SEQ ID NO: 2 Amino acid sequence of the alpha chain variable domain of an exemplary TCR. CDRs (CDR1, CDR2 and CDR3) are underlined and are designated SEQ ID NO: 3, 4 and 5 respectively, framework regions (FR1, FR2, FR3 and FR4) are in italics and are designated SEQ ID NO: 27, 6, 7 and 28 respectively. This sequence contains a N24Q mutation (double underlined), which removes an N-linked glycosylation site.

GDAKTTQPNSMESNEEEPVHLPC [symbol] HSTISGTDYIHWYROLPSQGPEYV
IHGLTSNVNNRMASLAIAEDRKSSTLILHRATLRDAAVYYCILILGHS
RLGNYIATFGKGTKLSVIP

SEQ ID NO: 8 Amino acid sequence of the TCRβ chain variable domain of an exemplary TCR. CDRs (CDR1, CDR2 and CDR3) are underlined and are designated SEQ ID NO: 9, 10 and 11 respectively, framework regions (FR1, FR2, FR3 and FR4) are in italics and are designated SEQ ID NO: 29, 12, 13 and 30 respectively.

DGGITQSPKYLFRKEGONVTLSCEQNLNHDAMYWYRQDPGQGLRLIYY
SQIMGDEQKGDIAEGYSVSREKKESFPLTVTSAQKNPTAFYLCASSWW
TGGASPIRFGPGTRLTVT

SEQ ID NO: 14 Amino acid sequence of the TCRα chain of an exemplary TCR. CDRs (CDR1, CDR2 and CDR3) are underlined and are designated SEQ ID NO: 3, 4 and 5 respectively, framework regions (FR1, FR2, FR3 and FR4) are in italics and are designated SEQ ID NO: 27, 6, 7 and 28 respectively. The constant region is shown in bold and is designated SEQ ID NO: 15. Within the constant region, a non-native cysteine residue is double underlined (at position 48 of the constant region) which was introduced to create an inter-chain disulphide bond. The sequence also contains

N24Q, N148Q, N182Q and N193Q substitutions (double underlined), which each remove an N-linked glycosylation site.

GDAKTTQPNSMESNEEEPVHLPC [symbol] HSTISGTDYIHWYRQLPSQGPEYV
IHGLTSNVNNRMASLAIAEDRKSSTLILHRATLRDAAVYYCILILGHSR
LGNYIATFGKGTKLSVIPNIQNPDPAVYQLRDSKSSDKSVCLFTDFDSQT [symbol]
VSQSKDSDVYITDK [symbol] VLDMRSMDFKSNSAVAWS [symbol] KSDFACANAF [symbol]
NSIIPEDT

SEQ ID NO: 16 Amino acid sequence of the TCRβ chain of an exemplary TCR. CDRs (CDR1, CDR2 and CDR3) are underlined and are designated SEQ ID NO: 9, 10 and 11 respectively, framework regions (FR1, FR2, FR3 and FR4) are in italics and are designated SEQ ID NO: 29, 12, 13 and 30 respectively. Constant region is shown in bold (no underline) and is designated SEQ ID NO: 19. Within the constant region, a non-native cysteine residue is shaded (at position 57 of the constant region) which was introduced to create an inter-chain disulphide bond. The sequence also contains an N184Q substitution (double underlined), which removes an N-linked glycosylation site.

DGGITQSPKYLFRKEGQNVTLSCEQNLNHDAMYWYRQDPGQGLRLIYYS
QIMGDEQKGDIAEGYSVSREKKESFPLTVTSAQKNPTAFYLCASSWWTG
GASPIRFGPGTRLTVTEDLKNVFPPEVAVFEPSEAEISHTQKATLVCLAT
GFYPDHVELSWWVNGKEVHSGV [symbol] TDPQPLKEQPAL [symbol] DSRYALSSRLRV
SATFWQDPRNHFRCQVQFYGLSENDEWTQDRAKPVTQIVSAEAWGRAD

SEQ ID NO: 17 An exemplary anti-CD3 scFv (T cell engaging immune effector domain) referred to herein as “U0”. The light chain variable domain (VL) is in italics and is designated SEQ ID NO: 31. The light chain CDRs (CDR1, CDR2 and CDR3) are underlined and are designated SEQ ID NO: 33, 34 and 35. The heavy chain variable domain (VH) is shown in bold and is designated SEQ ID NO: 32. The heavy chain CDRs (CDR1, CDR2 and CDR3) are underlined and are designated SEQ ID NO: 36, 37 and 38. A glycine-serine linker, linking the VL and VH, is shown in plain text and is designated SEQ ID NO: 39.

AIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIKGGGGSGGGGSGGGGSGGGGSGGGSEVQLVESGGGLVQPGGSLR
LSCAASGYSFTGYTMNWVRQAPGKGLEWVALINPYKGVSTYNQKFKDRFT
ISVDKSKNTAYLQMNSLRAEDTAVYYCARSGYYGDSDWYFDVWGQGTLVT
VSS

SEQ ID NO: 40 Another exemplary anti-CD3 scFv (T cell engaging immune effector domain) referred to herein as “U28”. This sequence is the same as SEQ ID NO: 17 above, except for two substitutions that are double underlined (T164A and I201F). The light chain variable domain (VL) is in italics and is designated SEQ ID NO: 31. The light chain CDRs (CDR1, CDR2 and CDR3) are underlined and are designated SEQ ID NO: 33, 34 and 35. The heavy chain variable domain (VH) is shown in bold and is designated SEQ ID NO: 41. The heavy chain CDRs (CDR1, CDR2 and CDR3) are underlined and are designated SEQ ID NO: 48, 37 and 38. A glycine-serine linker, linking the VL and VH, is shown in plain text and is designated SEQ ID NO: 39.

AIQMTQSPSSLSASVGDRVTITCRASQDIRNYLNWYQQKPGKAPK
LLIYYTSRLESGVPSRFSGSGSGTDYTLTISSLQPEDFATYYCQQ
GNTLPWTFGQGTKVEIKGGGGSGGGGSGGGGSGGGGSGGGSEVQL
VESGGGLVQPGGSLRLSCAASGYSFTGYAMNWVRQAPGKGL
EWVALINPYKGVSTYNQKFKDRFTESVDKSKNTAYLQMNSL
RAEDTAVYYCARSGYYGDSDWYFDVWGQGTLVTVSS

SEQ ID NO: 54 Human IgG1 Fc region (CH2 and CH3 domains), unmodified

APELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

SEQ ID NO: 42 An exemplary IgG1 Fc region sequence. This sequence has four substitutions, double underlined, relative to the above unmodified IgG1 Fc sequence (SEQ ID NO: 54). These are an N297G substitution for inhibiting binding to FcγR as well as T366S, L368A, and Y407V substitutions (hole-forming substitutions) for enhancing dimerization with another Fc region (e.g., SEQ ID NO: 43) containing a T366W substitution (knob-forming substitution). The numbering of the substitutions in this sequence is according to the EU numbering scheme.

APELLGGPSVFLFPPKPKDTLMISRTPEVTCVVVDVSHEDPEVKENWYVD
GVEVHNAKTKPREEQYGSTYRVVSVLTVLHQDWLNGKEYKCKVSNKALPA
PIEKTISKAKGQPREPQVYTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGK

SEQ ID NO: 43 Another exemplary IgG1 Fc region sequence. This sequence has two substitutions, double underlined, relative to the above unmodified IgG1 Fc sequence (SEQ ID NO: 54). These are an N297G substitution for inhibiting binding to FcγR as well as a T366W substitution (knob-forming substitution) for enhancing dimerization with another Fc region (e.g., SEQ ID NO: 42) containing T366S, L368A, and Y407V substitutions (hole-forming substitutions). The numbering of the substitutions in this sequence is according to the EU numbering scheme.

APELLGGPSVELFPPKPKDTLMISRTPEVTCVVVDVSHEDPEVKENWYVD
GVEVHNAKTKPREEQYGSTYRVVSVLTVLHQDWLNGKEYKCKVSNKALPA
PIEKTISKAKGQPREPQVYTLPPSRDELTKNQVSLWCLVKGFYPSDIAVE
WESNGQPENNYKTTPPVLDSDGSFFLYSKLTVDKSRWQQGNVFSCSVMHE
ALHNHYTQKSLSLSPGK

SEQ ID NO: 57 An exemplary IgG1 Fc region sequence is shown below. This sequence has seven substitutions (bold) relative to the above unmodified IgG1 Fc sequence (SEQ ID NO: 54). These are an N297G substitution for inhibiting binding to FcγR, T366S, L368A, and Y407V substitutions (hole-forming substitutions) for enhancing dimerization with another Fc region (e.g., SEQ ID NO: 58) containing a T366W substitution (knob-forming substitution), and the substitutions M252Y, S254T and T256E, to increase binding to FcRn. The numbering of the substitutions in this sequence is according to the EU numbering scheme.

APELLGGPSVFLFPPKPKDTLYITREPEVTCVVVDVSHEDPEVKENWYVD
GVEVHNAKTKPREEQYGSTYRVVSVLTVLHQDWLNGKEYKCKVSNKALPA
PIEKTISKAKGQPREPQVYTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGK

SEQ ID NO: 58 A further exemplary IgG1 Fc region sequence is shown below. This sequence has five substitutions (bold) relative to the above unmodified IgG1 Fc sequence (SEQ ID NO: 54). These are an N297G substitution for inhibiting binding to FcγR, a T366W substitution (knob-forming substitution) for enhancing dimerization with another Fc region (e.g., SEQ ID NO: 57) containing T366S, L368A, and Y407V substitutions (hole-forming substitutions), and the substitutions M252Y, S254T and T256E, to increase binding to FcRn. The numbering of the substitutions in this sequence is according to the EU numbering scheme.

APELLGGPSVFLFPPKPKDTLYITREPEVTCVVVDVSHEDPEVKENWYVD
GVEVHNAKTKPREEQYGSTYRVVSVLTVLHQDWLNGKEYKCKVSNKALPA
PIEKTISKAKGQPREPQVYTLPPSRDELTKNQVSLWCLVKGFYPSDIAVE
WESNGQPENNYKTTPPVLDSDGSFFLYSKLTVDKSRWQQGNVFSCSVMHE
ALHNHYTQKSLSLSPGK

SEQ ID NO: 44 An exemplary IgG1 hinge sequence (containing a C to S substitution at position 5, numbered according to SEQ ID NO: 44, relative to the native human IgG1 sequence):

EPKSSDKTHTCPPCP

SEQ ID NO: 52 A truncated IgG1 hinge sequence:

DKTHTCPPCP

SEQ ID NO: 53 An IgG4 hinge sequence:

ESKYGPPCPSCP

SEQ ID NO: 45 A complete amino acid sequence of an exemplary multi-domain, single-chain binding molecule named “mol093v11”. The T cell engaging immune effector domain (underlined) is the anti-CD3 scFv sequence provided in SEQ ID NO: 17 (“U0”). The pMHC binding domain is double underlined and comprises the TCRβ chain sequence (which in this case is “VC1”) provided in SEQ ID NO: 16 (double underlined, plain text) and the TCRα chain sequence (which in this case is “VC2”) provided in SEQ ID NO: 14 (double underlined, bold text). The half-life extending domain is an Fc domain which is a dimer formed between the Fc region sequence provided in SEQ ID NO: 42 (italics), which in this case is the FC1 region, and the Fc region sequence provided in SEQ ID NO: 43 (italics and bold), which in this case is the FC2 region.

AIQMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKGGGGSGGGGSGGGGSGG
GGSGGGSEVQLVESGGGLVQPGGSLRLSCAASGYSFTGYTMNWVRQAPGKGLEWVALINPYK
GVSTYNQKFKDRFTISVDKSKNTAYLQMNSLRAEDTAVYYCARSGYYGDSDWYFDVWGQGTL
VTVSSGGGGSDGGITQSPKYLFRKEGQNVTLSCEQNLNHDAMYWYRQDPGQGLRLIYYSQIM
GDEQKGDIAEGYSVSREKKESFPLTVISAQKNPTAFYLCASSWWTGGASPIREGPGTRLTVT
EDLKNVEPPEVAVFEPSEAEISHTQKATLVCLATGFYPDHVELSWWVNGKEVHSGVCTDPQP
LKEQPALQDSRYALSSRLRVSATFWQDPRNHERCQVQFYGLSENDEWTQDRAKPVTQIVSAE
AWGRADGGGSGGGGEPKSSDKTHTCPPCPAPELLGGPSVFLFPPKPKDTLMISRTPEVTCVV
VDVSHEDPEVKFNWYVDGVEVHNAKTKPREEQYGSTYRVVSVLTVLHQDWLNGKEYKCKVSN
KALPAPIEKTISKAKGQPREPQVYTLPPSRDELTKNQVSLSCAVKGFYPSDIAVEWESNGQP
ENNYKTTPPVLDSDGSFFLVSKLTVDKSRWQQGNVFSCSVMHEALHNHYTQKSLSLSPGKGG
GSGGGGGDAKTTQPNSMESNEEEPVHLPCQHSTISGTDYIHWYRQLPSQGPEYVIHGLTSNV
NNRMASLAIAEDRKSSTLILHRATLRDAAVYYCILILGHSRLGNYIATFGKGTKLSVIPNIQ
NPDPAVYQLRDSKSSDKSVCLFTDFDSQTQVSQSKDSDVYITDKCVLDMRSMDFKSNSAVAW
SQKSDFACANAFQNSIIPEDTGGGSGGGGEPKSSDKTHTCPPCPAPELLGGPSVFLFPPKPK
DTLMISRTPEVTCVVVDVSHEDPEVKENWYVDGVEVHNAKTKPREEQYGSTYRVVSVLTVLH
QDWLNGKEYKCKVSNKALPAPIEKTISKAKGQPREPQVYTLPPSRDELTKNQVSLWCLVKGF
YPSDIAVEWESNGQPENNYKTTPPVLDSDGSFFLYSKLTVDKSRWQQGNVFSCSVMHEALHN
HYTQKSLSLSPGK

SEQ ID NO: 46 A complete amino acid sequence of an exemplary multi-domain, single-chain binding molecule named “mol093v9”. The T cell engaging immune effector domain (underlined) is the anti-CD3 scFv sequence provided in SEQ ID NO: 40 (“U28”). The pMHC binding domain is double underlined and comprises the TCRβ chain sequence (which in this case is “VC1”) provided in SEQ ID NO: 16 (double underlined, plain text) and the TCRα chain sequence (which in this case is “VC2”) provided in SEQ ID NO: 14 (double underlined, bold text). The half-life extending domain is an Fc domain which is a dimer formed between the Fc region sequence provided in SEQ ID NO: 42 (italics), which in this case is the FC1 region, and the Fc region sequence provided in SEQ ID NO: 43 (italics and bold), which in this case is the FC2 region.

AIQMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKGGGGSGGGGSGGGGSGG
GGSGGGSEVQLVESGGGLVQPGGSLRLSCAASGYSFTGYAMNWVRQAPGKGLEWVALINPYK
GVSTYNQKFKDRFTFSVDKSKNTAYLQMNSLRAEDTAVYYCARSGYYGDSDWYFDVWGQGTL
VTVSSGGGGSDGGITQSPKYLFRKEGQNVTLSCEQNLNHDAMYWYRQDPGQGLRLIYYSQIM
GDEQKGDIAEGYSVSREKKESFPLTVTSAQKNPTAFYLCASSWWTGGASPIREGPGTRLTVT
EDLKNVEPPEVAVFEPSEAEISHTQKATLVCLATGFYPDHVELSWWVNGKEVHSGVCTDPQP
LKEQPALQDSRYALSSRLRVSATFWQDPRNHFRCQVQFYGLSENDEWTQDRAKPVTQIVSAE
AWGRADGGGSGGGGEPKSSDKTHTCPPCPAPELLGGPSVFLFPPKPKDTLMISRTPEVTCVV
VDVSHEDPEVKFNWYVDGVEVHNAKTKPREEQYGSTYRVVSVLTVLHQDWLNGKEYKCKVSN
KALPAPIEKTISKAKGQPREPQVYTLPPSRDELTKNQVSLSCAVKGFYPSDIAVEWESNGQP
ENNYKTTPPVLDSDGSFFLVSKLTVDKSRWQQGNVFSCSVMHEALHNHYTQKSLSLSPGKGG
GSGGGGGDAKTTQPNSMESNEEEPVHLPCQHSTISGTDYIHWYRQLPSQGPEYVIHGLTSNV
NNRMASLAIAEDRKSSTLILHRATLRDAAVYYCILILGHSRLGNYIATFGKGTKLSVIPNIQ
NPDPAVYQLRDSKSSDKSVCLFTDFDSQTQVSQSKDSDVYITDKCVLDMRSMDFKSNSAVAW
SQKSDFACANAFQNSIIPEDTGGGSGGGGEPKSSDKTHTCPPCPAPELLGGPSVFLFPPKPK
DTLMISRTPEVTCVVVDVSHEDPEVKENWYVDGVEVHNAKTKPREEQYGSTYRVVSVLTVLH
QDWLNGKEYKCKVSNKALPAPIEKTISKAKGQPREPQVYTLPPSRDELTKNQVSLWCLVKGF
YPSDIAVEWESNGQPENNYKTTPPVLDSDGSFFLYSKLTVDKSRWQQGNVFSCSVMHEALHN
HYTQKSLSLSPGK

SEQ ID NO: 55 A complete amino acid sequence of an exemplary multi-domain, single-chain binding molecule named “a40b23U28-mol93”. The T cell engaging immune effector domain (underlined) is the anti-CD3 scFv sequence provided in SEQ ID NO: 40 (“U28”). The pMHC binding domain in this molecule binds to a human PIWIL1 (PIWI-like protein 1) peptide-MHC complex. The pMHC binding domain is double underlined and comprises a TCRβ chain sequence (which in this case is “VC1”) (double underlined, plain text) and a TCRα chain sequence (which in this case is “VC2”) (double underlined, bold text). The half-life extending domain is an Fc domain which is a dimer formed between the Fc region sequence provided in SEQ ID NO: 42 (italics), which in this case is the FC1 region, and the Fc region sequence provided in SEQ ID NO: 43 (italics and bold), which in this case is the FC2 region.

AIQMTQSPSSLSASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKGGGGSGGGGSGGGGSGG
GGSGGGSEVQLVESGGGLVQPGGSLRLSCAASGYSFTGYAMNWVRQAPGKGLEWVALINPYK
GVSTYNQKFKDRFTFSVDKSKNTAYLQMNSLRAEDTAVYYCARSGYYGDSDWYFDVWGQGTL
VTVSSGGGGSEAGVAQSPRYKIIEKRQSVAFWCNPISGHGTLYWYQQILGQGPKLLIQFHEN
GVVDDSQLPKDRFSAERLKGVDSTLKIQPAKLEDSAVYLCASSVDWVGDGERQYFGPGTRLL
VLEDLKNVEPPEVAVFEPSEAEISHTQKATLVCLATGFYPDHVELSWWVNGKEVHSGVCTDP
QPLKEQPALQDSRYALSSRLRVSATFWQDPRNHERCQVQFYGLSENDEWTQDRAKPVTQIVS
AEAWGRADGGGSGGGGEPKSSDKTHTCPPCPAPELLGGPSVFLFPPKPKDTLMISRTPEVTC
VVVDVSHEDPEVKFNWYVDGVEVHNAKTKPREEQYGSTYRVVSVLTVLHQDWLNGKEYKCKV
SNKALPAPIEKTISKAKGQPREPQVYTLPPSRDELTKNQVSLSCAVKGFYPSDIAVEWESNG
QPENNYKTTPPVLDSDGSFFLVSKLTVDKSRWQQGNVFSCSVMHEALHNHYTQKSLSLSPGK
GGGSGGGGLAKTTQPISMDSYEGQEVNITCSHNYIAANDFITWYQQFPSQGPRFFIQGYKTN
VQNEVASLFIPADRKSSTLSLPRVSLSDTAVYYCLAWGGTDLLPFGTGTRLQVFPNIQNPDP
AVYQLRDSKSSDKSVCLFTDFDSQTQVSQSKDSDVYITDKCVLDMRSMDFKSNSAVAWSQKS
DFACANAFQNSIIPEDTGGGSGGGGEPKSSDKTHTCPPCPAPELLGGPSVFLFPPKPKDTLM
ISRTPEVTCVVVDVSHEDPEVKFNWYVDGVEVHNAKTKPREEQYGSTYRVVSVLTVLHQDWL
NGKEYKCKVSNKALPAPIEKTISKAKGQPREPQVYTLPPSRDELTKNQVSLWCLVKGFYPSD
IAVEWESNGQPENNYKTTPPVLDSDGSFFLYSKLTVDKSRWQQGNVFSCSVMHEALHNHYTQ
KSLSLSPGK

SEQ ID NO: 56 Amino acid sequence of the PIWIL1 peptide bound (when in complex with MHC) by a40b23U28-mol93.

SLSNRLYYL

### Additional Linker Sequences:

(SEQ ID NO: 18)
GGGGS,
(SEQ ID NO: 20)
GGGSG,
(SEQ ID NO: 21)
GGSGG,
(SEQ ID NO: 22)
GSGGG,
(SEQ ID NO: 23)
GSGGGP,
(SEQ ID NO: 24)
GGEPS,
(SEQ ID NO: 25)
GGEGGGP,
(SEQ ID NO: 26)
GGEGGGSEGGGS,
(SEQ ID NO: 47)
GGGSGGGG,
(SEQ ID NO: 39)
GGGGSGGGGSGGGGSGGGGSGGGS,
(SEQ ID NO: 49)
GGGGSGGGGSGGGGS,
(SEQ ID NO: 50)
EAAAK
and
(SEQ ID NO: 51)
EAAAKEAAAKEAAAK.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the purview of this application and scope of the appended claims.

### Example 1—Multidomain Molecules with Improved Potency

Multidomain molecules comprising a TCR-anti-CD3 fusion protein and incorporating a half-life extending Fc domain have been described previously WO 2020/157211 and shown to be functional. However, it was subsequently found that such molecules have substantially reduced ability to activate

T cells in vitro relative to the non Fc-fused format of the molecule and therefore were not considered optimal for therapeutic use. Further engineering was carried out to identify novel molecular formats with improved therapeutic properties.

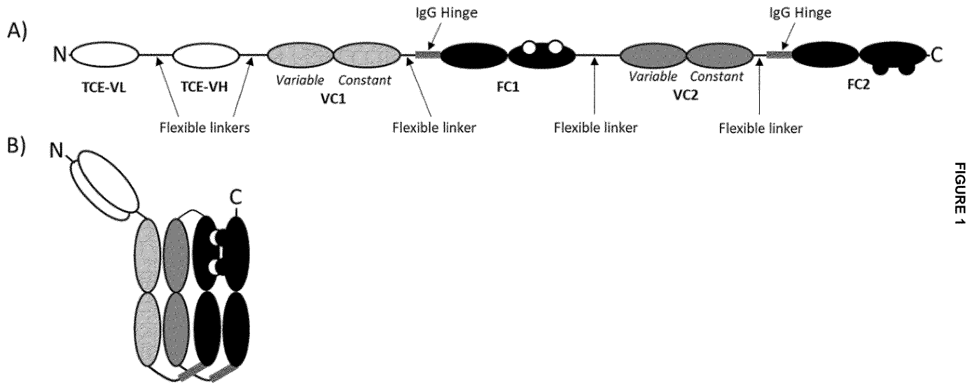

A multidomain molecule was constructed in which each of the functional domains was arranged on a single polypeptide chain. FIG. 1a shows a schematic representation of the domain arrangement and FIG. 1b shows a hypothetical representation of the folded structure the molecule.

In a first example the TCR domains of the multidomain single chain molecule were designed to recognize the HLA-A*02 restricted peptide SLLQHLIGL (SEQ ID NO: 1) derived from PRAME, as described previously (WO 2018/234319). The ability of this molecule to drive T cell activation in the presence of antigen positive cancer cells was investigated using an ELISpot assay, using IFNγ as a read out of T cell activation. As a comparison, the same TCR was used to construct a multidomain molecule using the previously disclosed format in WO 2020/157211 and tested alongside the single chain format presented in FIG. 1.

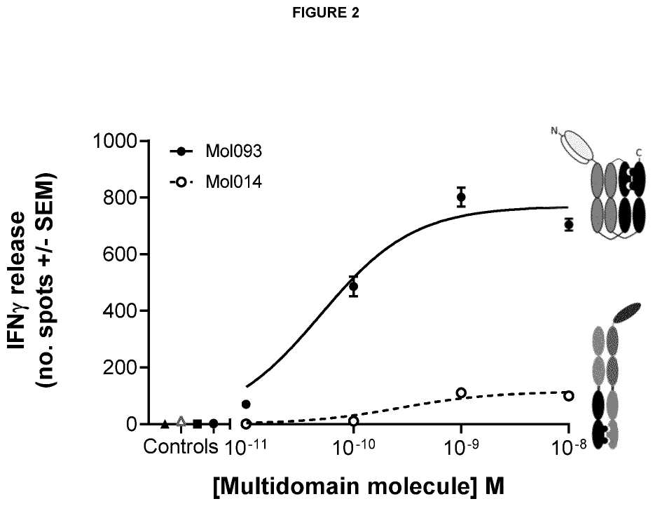

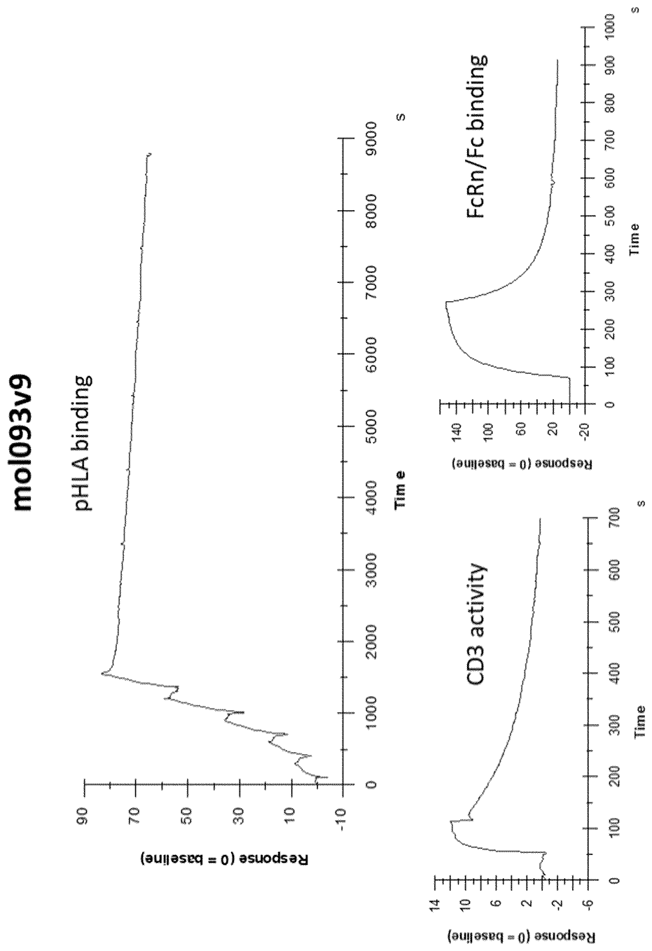

The data shown in FIG. 2 demonstrate that the single chain molecule is able to drive a substantially improved T cell response against antigen positive cancer cells than the previously disclosed format.

A further 37 molecular formats with alternative domain arrangements were made and tested for in vitro potency. None of these formats performed better than the molecule depicted in FIG. 1. Little or no response was observed with 17 of the formats; a low-level response was observed with 15 formats and an intermediate level response was observed with 2 formats. The three remaining formats demonstrated increased cross reactivity against antigen negative cell lines in addition to low potency against antigen positive cells.

### Example 2—Preparation of Single Chain Multidomain Molecules Targeting PRAME

Two multidomain molecules (termed mol093v9 and mol093v11) were prepared using the format shown in FIG. 1. The TCR regions of both molecules were designed to recognize the HLA-A*02 restricted peptide SLLQHLIGL derived from PRAME. The two molecules differ in the amino acid sequence of the antiCD3 scFv fragment. The full amino acid sequence of mol093v9 and mol093v11 is provided in SEQ ID NOs: 46 and 45 respectively.

### Expression

Mol093v9 and mol093v11 were expressed in Cho cells using the Thermo ExpiCHO™ transient expression protocol. Briefly, cultured cells were diluted to a concentration of 6×10⁶ prior to transfection. Cells were harvested on day 14 post transfection, with temperature shift to 32° C. at day 1 post transfection. Feed additions were performed on day 1 and day 5 post transfection. Clarification was performed with two successive centrifugation steps, at 300×g and 17,500×g. The resulting supernatant was passed through 0.45 μm and 0.2 μm membrane filters.

### Purification

Clarified supernatant was purified by Protein A followed by size exclusion chromatography steps. A 15 cm bed height MabSelect Extra Protein A resin column was prepared. The column was loaded with 50 column volumes of supernatant with elution using a sodium citrate buffer at pH 3.0. Eluted product was neutralised with the addition of 2M Tris after three column volumes had been collected and filtered through a 0.2 μm membrane filter. Protein A eluate was concentrated using tangential flow filtration (Pellicon® XL50 with Ultracel® 30 kDa Membrane) to at least 2 mg/mL before loading on to a HiLoad 26/600 Superdex SEC resin. The column was loaded at 5% column volume. Product was eluted into phosphate citrate buffer, with relevant fractions filtered through a 0.22 μm membrane filter.

### Yield

The concentration of purified material was measured by absorbance at 280 nm using a Nanodrop spectrophotometer.

The calculated yield per litre of supernatant was 9.6 mg/L for mol093v9 and 17 mg/L mol093v11.

### Stability

Molecule stability was assessed by SEC UPLC following a freeze/thaw cycle, and/or under conditions of i) thermal stress and ii) agitation, for 14 days. The results are provided in the table immediately below, which show mol093v11 monomer purity under indicated conditions. In each case monomer purity was considered acceptable.

| | Mol093 v11 (U0) | | | Mol093 v9 (U28) | | |
|---|---|---|---|---|---|---|
| | Total | Total | Total | Total | Total | Total |
| Sample condition | HMW | monomer | LMW | HMW | monomer | LMW |
| Control (5 °C) | 3.3 | 96.7 | <0.1 | — | — | — |
| Freeze/thaw | 3.5 | 96.5 | <0.1 | — | — | — |
| Control (5 °C) | 4.6 | 95.3 | 0.1 | 3.0 | 97.0 | <0.1 |
| Agitation | 5.6 | 94.3 | 0.1 | 3.1 | 96.8 | <0.1 |
| 30 °C | 8.2 | 91.6 | 0.2 | 5.5 | 94.4 | 0.1 |

### Example 3—Binding Affinity and Kinetics of Multidomain Molecules Targeting PRAME

To verify that the TCR and anti-CD3 portions of the molecule bind to respective target molecules, single cycle kinetics was performed by Surface Plasmon Resonance (SPR) on a T200 BIAcore, followed by a single injection of CD3 (γε).

### Method

The chip used was from a Serie-S Biotin CAPture Kit (Cytiva). Running buffer was phosphate buffer saline (PBS) pH7.2 with P20 at 0.005%. The chip was regenerated with three consecutive injections of a solution of guanidine hydrochloride 8M (GuHCl) and sodium hydroxide 1M (NaOH) with a ratio 3+1. Flow rate was 20 μL/minute, contact time 120 sec. The chip was activated with the biotin CAPture reagent diluted 1:16 with PBS+P20. Flow rate was 2 μL/minute for 300 sec. The amount captured was 1600 Response Unit (RU). Biotinylated pHLA was injected at 10 μg/mL, flow rate 10 μL/minute for 120 sec.

For single cycle kinetic analysis serial dilutions of mol093v9 and mol093v11 were injected (top conc=15 nM) at a flow rate of 60 μL/minute with 200 sec dissociation in between injection and 7200 seconds dissociation for the 5th injection.

CD3 (γε) was subsequently injected at a concentration of 300 nM and a flow rate 10 μL/minute for 60 seconds.

For FcRn capture, the flow cells were prime with PBS+P20 0.005% pH6.0. Injection of biotinylated FcRn was carried out at 5 μg/mL, flow rate 2 μL/minute for 120 seconds. Post-injection of the FcRn the biotin at 5 μM is injected on all flow cells at 10 L/minute for 120 seconds. The amount FcRn was 450 RU.

Mol093v9 or mol093v11 were injected at 15 nM, flow rate 10 μL/minute for 300 seconds and a dissociation of 600 seconds. The response was 152 RU and 163 RU for mol093v9 and mol093v11 respectively.

Kinetic parameters were calculated using the manufacturer's software. The dissociation phase was fitted to a single exponential decay equation enabling calculation of half-life. The equilibrium constant K_{D} was calculated from K_{off}/Kₒₙ.

### Results

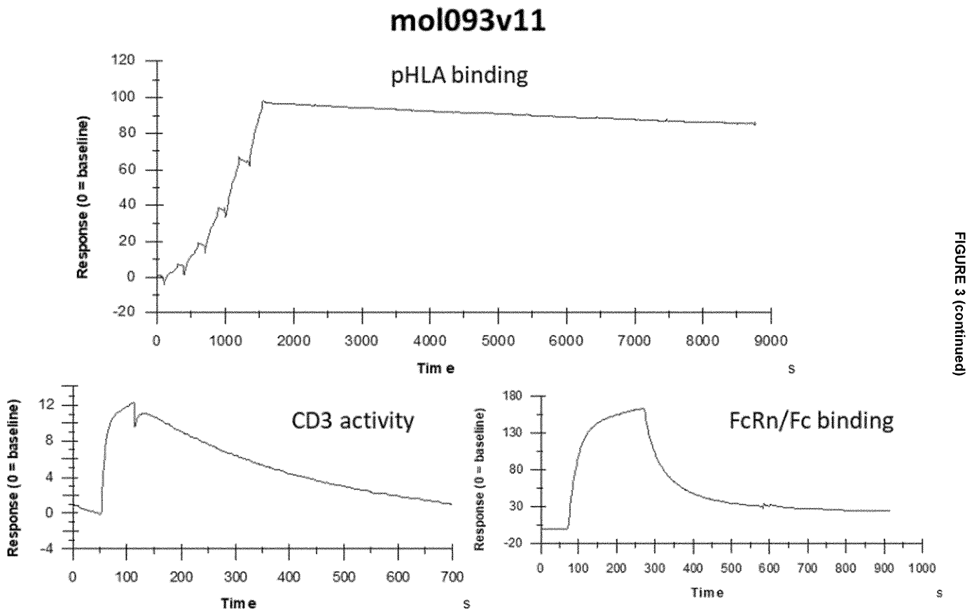

Mol093v9 and mol093v11 demonstrated picomolar affinity for pHLA complex along with a high level of CD3 activity and binding to FcRn. Data are shown in FIG. 3 and in the binding parameters are summarised in the table immediately below.

| | ka | kd | KD | KD | | CD3 | FcRn |
|---|---|---|---|---|---|---|---|
| | (1/Ms) | (1/s) | (M) | (pM) | t½(hr) | activity (%) | binding |
| mol93v9 | 3.55E+05 | 2.56E−05 | 7.20E−11 | 72.02 | 7.54 | 83.5% | yes |
| mol93v11 | 2.86E+05 | 1.82E−05 | 6.38E−11 | 63.77 | 10.57 | 84.1% | yes |

### Example 4—Pharmacokinetics

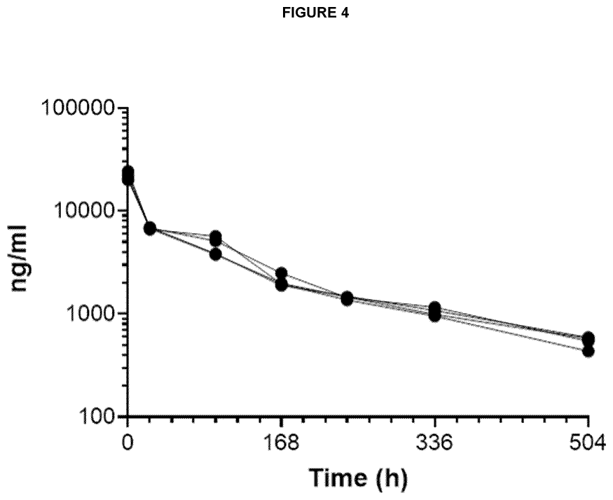

Pharmacokinetic properties were assessed in Tg32 SCID mice. Test article was dosed by IV bolus at 1 mg/Kg, 4 mice per compound, with serial sampling of blood over a 21 day period. Sample was detected in serum by electrochemiluminescent immunoassay, with capture on biotinylated PRAME peptide-HLA, and detection with sulfo-tagged anti-scFv antibody. FIG. 4 shows serum concentration over time for 4 individual mice.PK parameters were extracted by non-compartmental analysis.

### Results

Terminal t_{1/2} of Mol93v9 was calculated to be 9 days. Results of the non-compartmental analysis are shown in the table immediately below.

| | Cₘₐₓ | Tₘₐₓ | T_{1/2} | SEM of | AUC_{inf} | Clearance |
|---|---|---|---|---|---|---|
| Molecule | (ng/ml) | (min) | (h) | T_{1/2} (h) | (h*ng/ml) | (ml/h/kg) |
| Mol093v9 | 21814 | 5 | 171 | 13.0 | 1515240 | 0.66 |

The results of 2-compartment modeling using NONMEM are shown in the table immediately below.

| | Mouse |
|---|---|
| Parameter | (25 g) |
| CL (mL/h) | 0.0211 |
| V_{c} (mL) | 1.24 |
| Q (mL/h) | 0.0358 |
| Vₚ (mL/kg) | 3.60 |
| Proportional | 25.8 |
| Error (%) | |
| t_{1/2α} (hr) | 13.2 |
| t_{1/2β} (hr) | 216 |

### Example 5—In Vitro T Cell Activation

Mol093v9 and Mol093v11 were assessed for their ability to mediate potent and specific activation of CD3+ T cells against cells presenting the SLLQHLIGL-HLA-A*02 complex. Interferon-γ (IFN-γ) release was used as a read out for T cell activation.

### Method

Assays were performed using a human IFN-γ ELISPOT kit (BD Biosciences) according to the manufacturer's instructions. Briefly, target cells were prepared at a density of 1×10⁶/ml in assay medium (RPMI 1640 containing 10% heat inactivated FBS and 1% penicillin-streptomycin-L-glutamine) and plated at 50,000 cells per well in a volume of 50 μl. Peripheral blood mononuclear cells (PBMC), isolated from fresh donor blood, were used as effector cells and plated at roughly 1:1 ratio with target cells in a volume of 50 μl (the exact number of PBMC used for each experiment is donor dependent and may be adjusted to produce a response within a suitable range for the assay). Fusion molecules were titrated down from 10 nM to give final concentrations represented (spanning the anticipated clinically relevant range) and added to the well in a volume of 50 μl.

Plates were prepared according to the manufacturer's instructions. Target cells, effector cells and fusion molecules were added to the relevant wells and made up to a final volume of 200 μl with assay medium. All reactions were performed in triplicate. Control wells were also prepared with the omission of fusion molecules. The plates were then incubated overnight (37° C./5% CO2). The next day the plates were washed three times with wash buffer (1×PBS sachet, containing 0.05% Tween-20, made up in deionised water). Primary detection antibody was then added to each well in a volume of 50 μl. Plates were incubated at room temperature for 2 hours prior to being washed again three times. Secondary detection was performed by adding 50 μl of diluted streptavidin-HRP to each well and incubating at room temperature for 1 hour and the washing step repeated. No more than 15 mins prior to use, one drop (20 μl) of AEC chromogen was added to each 1 ml of AEC substrate and mixed and 50 μl added to each well. Spot development was monitored regularly, and plates were washed in tap water to terminate the development reaction. The plates were allowed to dry at room temperature for at least 2 hours prior to spot counting using a CTL analyser with Immunospot software (Cellular Technology Limited).

In this example, the following cells lines were used as target cells:

### Antigen Positive:

Mel624—human melanoma cell line
NCI-H1755—non-small cell lung cancer (NSCLC) cell line
OV56—ovarian serous carcinoma cell line
THP-1—acute monocytic leukemia cell line
NCI-H1703—lung squamous cell carcinoma cell line
COV318—ovarian serous carcinoma cell line

### Antigen Negative:

TY-KNU—ovarian serous adenocarcinoma (HLA-A*02-ve; PRAME-ve)
NCI-H1693—non-small cell lung cancer (NSCLC) cell line (HLA-A*02+ve; PRAME-ve)

### Results

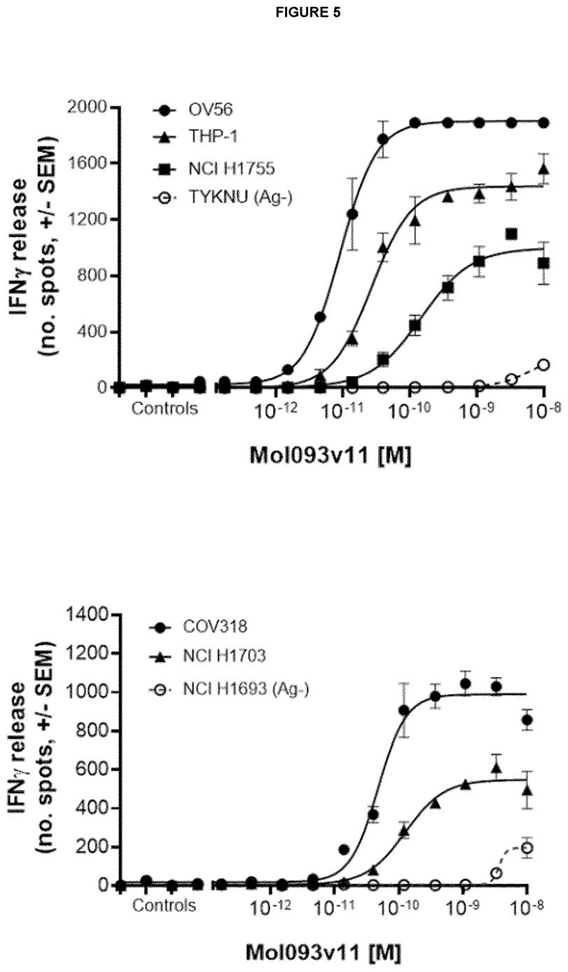

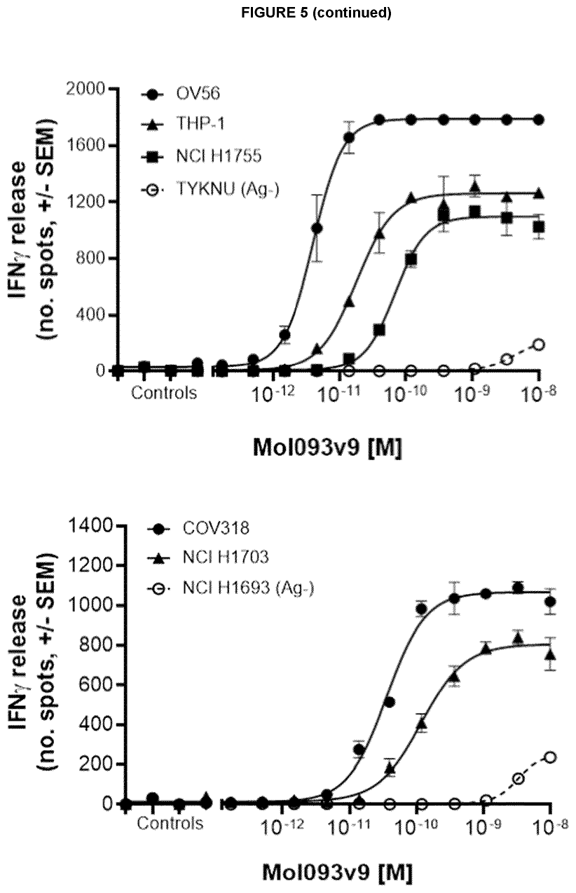

Mol093v9 and mol093v11 demonstrated potent activation of T cells in the presence of various antigen positive cancer cells. EC₅₀ values were calculated from the data and were obtained using PBMCs from two separate donors. T cell activation EC₅₀ Values for both donors are shown in the table immediately below. FIG. 5 shows data obtained from donor 1. Limited responses were observed in antigen negative cell lines.

| | |
|---|---|
| Mol093v11 EC₅₀ | Mol093v9 EC₅₀ |

| Cell line | Donor 1 | Donor 2 | Donor 1 | Donor 2 |
|---|---|---|---|---|
| NCI-H1755 | 147.6 | 64.1 | 69.1 | 62.9 |
| NCI-H1703 | 126.6 | 231.8 | 120.0 | 117.0 |
| OV56 | 9.2 | 10.8 | 4.0 | 5.1 |
| COV318 | 47.7 | 37.1 | 36.3 | 29.8 |
| THP-1 | 27.0 | 19.7 | 18.0 | 14.7 |

### Comparative Data

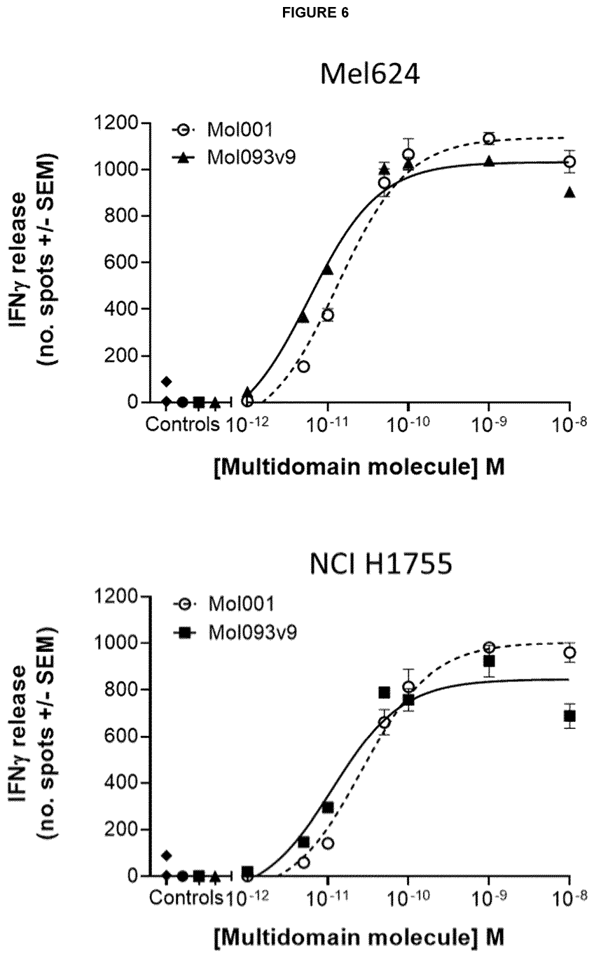

T cell activation driven by mol093v9 was compared directly with an alternative molecule targeting the same PRAME peptide, but which does not include the half-life extending Fc domain. Such molecules are described in WO 2018/234319 and U.S. Pat. No. 11,427,624, the contents of each which are herein incorporated by reference. ELISPot assays were carried out as described above. FIG. 6 shows that both molecules drive a similarly potent T cell response.

### Example 6—T Cell Killing

Mol093v9 and Mol093v11 were assessed for their ability to mediate potent and specific killing of antigen positive cancer cells.

### Method

Assays were performed either using the xCELLigence platform with appropriate 96 well plates for impedance reading (xCELLigence E-plate 96 PET part number 300600900) or the Incucyte live cell imagining platform with the CellPlayer 96-well Caspase-3/7 apoptosis assay kit (Essen BioScience, Cat. No. 4440), and carried out according to the manufacturer's instructions. Target cells were plated at respective optimal density (number of targets added per well varied for each cell line and had been previously titrated to determine optimal conditions) and incubated overnight to allow them to adhere. Test molecules were prepared at various concentrations and 50 μl of each was added to the relevant well such that final concentrations were between 100 fM and 10 nM. Effector cells were used at an effector target cell ratio of 10:1 and plated in 50 μl. A control sample without fusion was also prepared along with samples containing either effector cells alone, or target cells alone. For the xCELLigence platform, final volume in plate were adjusted to 200 μl using assay medium. The percentage of cytolysis was determined using the normalised Cell Index (impedance measurement). For the Incucyte platform, NucView assay reagent was made up at 30 UM and 25 μl added to every well and the final volume brought to 150 μl (giving 5 UM final conc). The number of apoptotic cells in each image was determined and recorded as apoptotic cells per mm2. In all cases, assays were performed in triplicate measurements were taken every 2 hours over 96 hours.

### Results

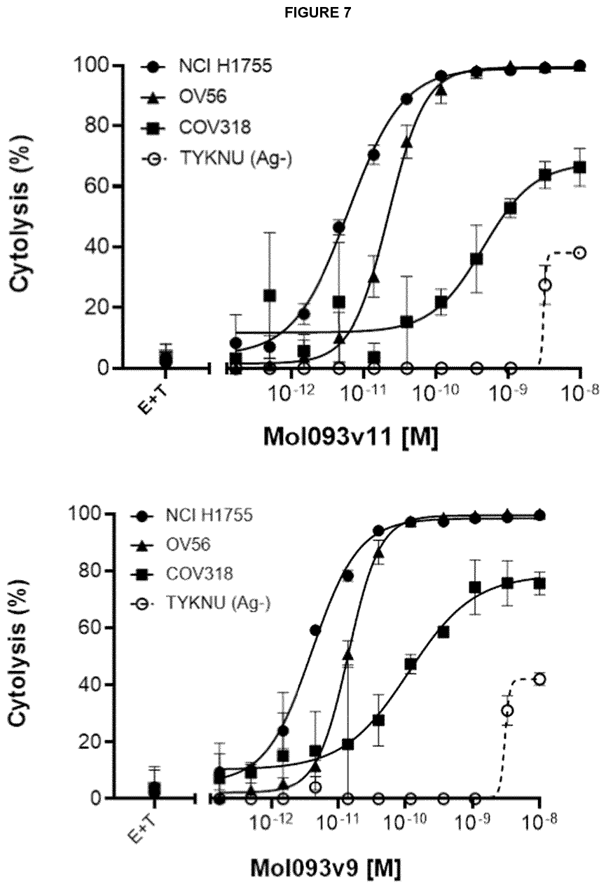

The data presented in FIG. 7 show real-time killing, as determined using the xCELLigence platform, of antigen positive cells in the presence of mol093v9 and mol093v11. EC₅₀ values are shown in the table immediately below and were in the low pM range. Limiting killing was detected of antigen negative cell lines.

| | Mol093v11 | Mol093v9 |
|---|---|---|
| Cell line | EC₅₀ (pM) | EC₅₀ (pM) |
| NCI-H1755 | 6.2 | 4.1 |
| NCI-H1703 | 16.7 | 12.0 |
| OV56 | 22.3 | 14.2 |
| COV318 | 460.2 | 111.8 |

### Comparative Data

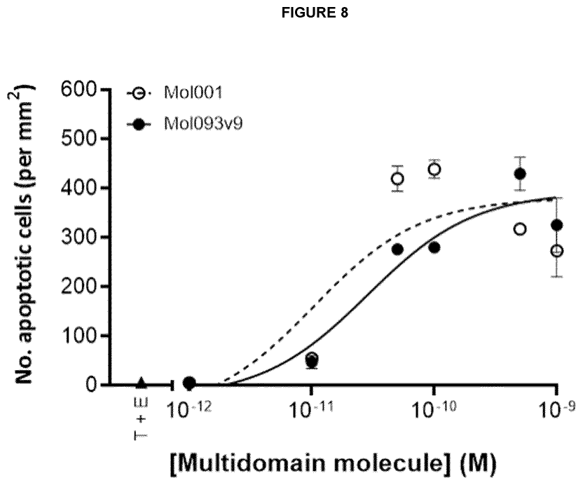

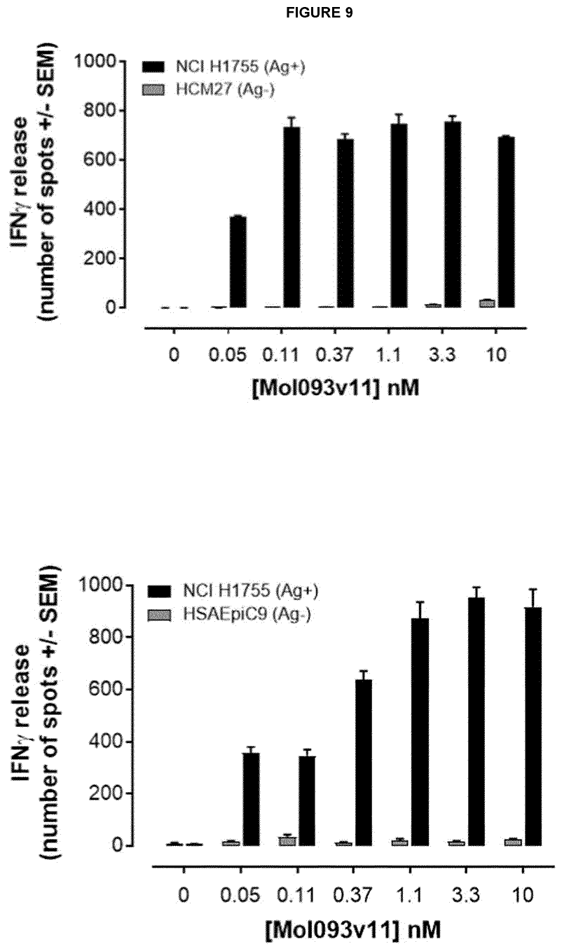

T cell activation driven by mol093v9 was compared directly with an alternative molecule targeting the same PRAME peptide, but which does not include the half-life extending Fc domain. Such molecules are described in WO 2018/234319. Killing assays were carried out using the Incucyte platform as described above. FIG. 8 shows that both molecules drive a similarly potent killing response.

### Example 7—Minimal Reactivity Against High Risk Normal Tissues

To demonstrate the specificity of mol093v9 and mol093v11 further testing was carried out using the same ELISPOT methodology as described above and a panel of normal cells derived from healthy human tissues as targets. Normal tissues included heart, lung, kidney and skin.

TCR-antiCD3 fusion molecules were tested at six different concentrations ranging from 50 μM to 10 nM against target normal cell lots co-cultured with PBMC from healthy donor. Control measurements were made using a sample without fusion molecule and a sample in which normal cells were replaced with NCI-H1755 (antigen positive) cells.

### Results

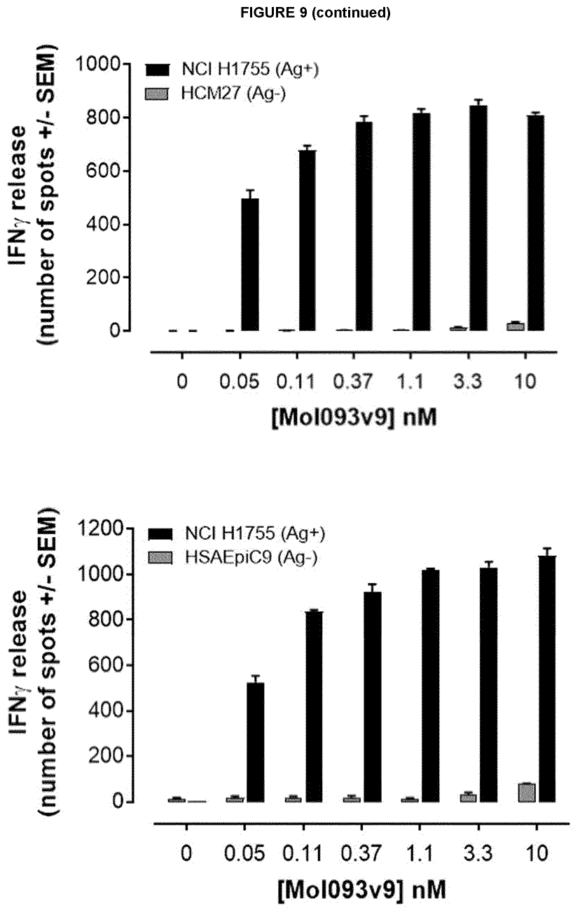

FIG. 9 shows data obtained with two normal cell lots (cardiac cells (HCM27) and lung epithelial cells (HSAEpiC9)) for one PBMC effector donor. Mimical T cell activation against normal cells was observed for concentrations of mol093v9 and mol093v11 up to and including 1.1 nM of fusion molecule.

### Comparative Data

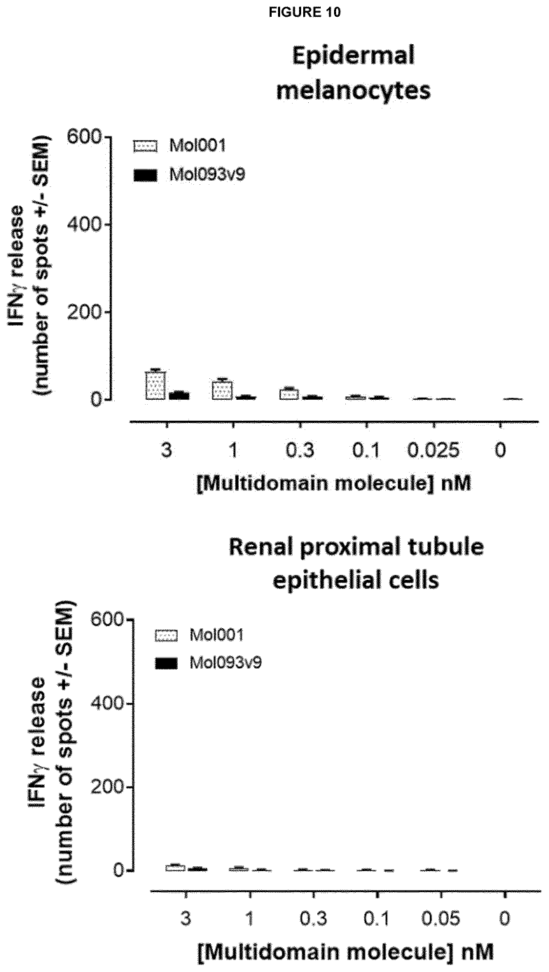

Normal cells reactivity for mol093v9 was compared directly with an alternative molecule targeting the same PRAME peptide, but which does not include the half-life extending Fc domain. Such molecules are described in WO 2018/234319. FIG. 10 shows that both molecules show a similar lack of reactivity against normal cells from skin (melanocytes) and kidney (renal proximal tubule).

### Example 8—Multidomain Single Chain Binding Molecules Targeting a PIWIL1 Peptide-MHC Complex demonstrate Potent and Specific T Cell Activation

A further multi-domain single-chain binding molecule was designed comprising a pMHC binding domain targeting a PIWIL1 peptide (SLSNRLYYL, SEQ ID NO: 56)-MHC complex, as opposed to mol093v9 and mol093v11 described above which bind to a PRAME peptide-MHC complex. The full sequence of the resulting molecule, termed “a40b23U28-mol93”, is provided in SEQ ID NO: 55. The sequence of a40b23U28-mol93 is identical to mol093v9 except for the TCRα and TCRβ variable domains. The TCRα variable and TCRβ variable domains of a40b23U28-mol93 correspond to SEQ ID NOs: 28 (“a40”) and 36 (“b23”), respectively, in GB application No. 2300226.4.

Binding of a40b23U28-mol93 to the SLSNRLYYL (SEQ ID NO: 56)-HLA-A*02 complex was determined using SPR as described in Example 3 above. The binding affinity (K_{D}) was 50 PM and the binding half life (t1/2) was 11.8 hours. These results were comparable to the equivalent TCR-antiCD3 fusion molecule without the half-life extending (i.e., Fc) domain.

The ability of a40b23U28-mol93 to drive T cell activation was determined by measuring IFNγ secretion using an ELISpot assay. Assays were performed using a human IFN-γ ELISPOT kit (BD Biosciences) according to the manufacturer's instructions. Peripheral blood mononuclear cells (PBMC), isolated from fresh donor blood, were used as effector cells. In this assay KATOIII (gastric carcinoma) and CL11 (colon carcinoma) were used as antigen positive target cells. NCI-H1755 was used as antigen negative cells. Data were plotted using PRISM software and EC₅₀ values were calculated from the curves.

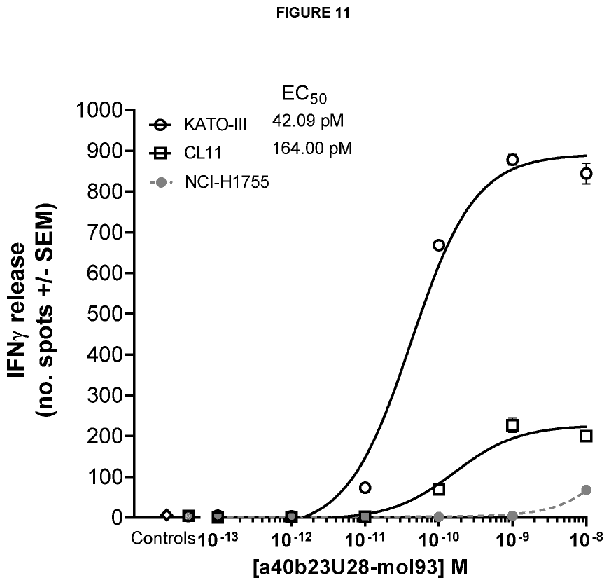

FIG. 11 shows that a40b23U28-mol93 gave an EC₅₀ value in the low pM range against the two antigen positive cells lines (42.1 μM for KATO-III and 164.0 μM for CL11), and little to no response (at concentrations of a40b23U28-mol93 less than 1 nM) in the presence of antigen negative cells. The equivalent TCR-antiCD3 fusion molecule without the half-life extending (i.e., Fc) domain had an EC₅₀ of 12.9 pM and 52.7 pM for KATO-III and CL11 cells respectively.

These data demonstrate that multi-domain single-chain binding molecules of the invention comprising a pMHC binding domain targeting a PIWIL1 peptide-MHC complex retain comparable high affinity and potency to the equivalent TCR-antiCD3 fusion molecule without the half-life extending (i.e., Fc) domain and retain specificity towards antigen positive cells.

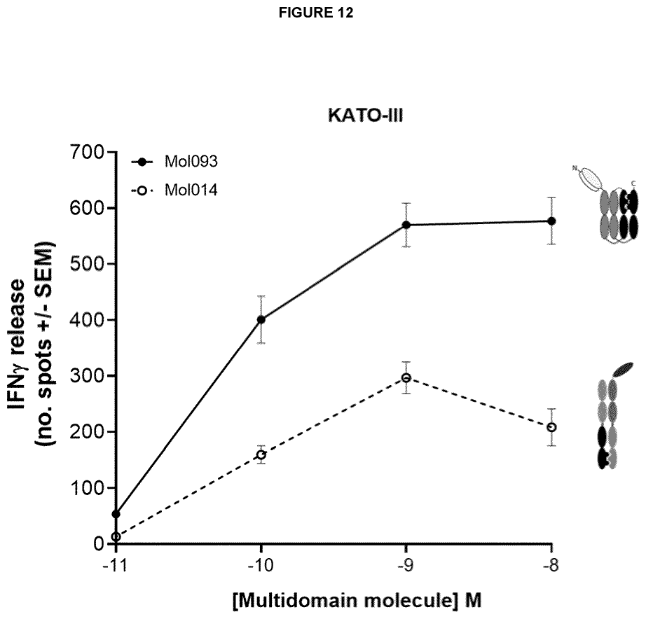

In a further experiment a40b23U28-mol93 was compared to an alternative multi-domain molecule format, termed a40b23U28-mol14. a40b23U28-mol14 has the same individual domain amino acid sequences as a40b23U28-mol93, except it is arranged in a two-chain format as depicted in FIG. 12. In this two-chain format, the first chain (left hand chain in FIG. 12) comprises, in the N- to C-terminal direction, a TCR alpha chain variable domain, a TCR alpha chain constant domain and an Fc region. The second chain (right hand chain in FIG. 12) comprises, in the N- to C-terminal direction, an anti-CD3 scFv, a TCR beta chain variable domain, a TCR beta chain constant domain and an Fc region. T cell activation against the KATOIII cell line was compared between the two molecules. As shown in FIG. 12, both molecules drive T cell activation; however, Mol93 gives a more potent response than Mol14.

The results described in this Example demonstrate that TCR variables domains that bind to a PIWIL1 peptide-MHC complex can be incorporated in the multi-domain single-chain binding molecule of the invention. In other words, regardless of the specific TCR sequences used (i.e., targeting PRAME peptide-MHC or PIWIL1 peptide-MHC), the multi-domain single-chain format was effective at extending half-life (via provision of the Fc domain) without significantly affecting affinity for the target or potency of T cell activation.

1. A multi-domain, single-chain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) a an immune cell engaging (ICE) domain; and
iii) a half-life extending domain comprising a first IgG Fc region (FC1) and a second IgG Fc region (FC2), wherein the FC1 region and FC2 region dimerise to form an Fc domain;
wherein the ICE domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of the FC2 region; and
wherein the pMHC binding domain and the T cell engaging immune effector domain are capable of binding to a pMHC complex and an immune cell respectively.

2. The multi-domain binding molecule of claim 1, wherein the immune cell engaging domain comprises:
i) an antibody light chain variable region (ICE-VL) and an antibody heavy chain variable region (ICE-VH); or
ii) a single domain antibody (ICE-SD).

3. The multi-domain binding molecule of claim 1 or claim 2, wherein the immune cell is a T cell and the immune cell engaging domain is selected from:
i) a T cell engaging immune effector domain; or
ii) a T cell engaging immune suppressor.

4. A multi-domain, single-chain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) a T cell engaging immune effector domain comprising an antibody light chain variable region (TCE-VL) and an antibody heavy chain variable region (TCE-VH); and
iii) a half-life extending domain comprising a first IgG Fc region (FC1) and a second IgG Fc region (FC2), wherein the FC1 region and FC2 region dimerise to form an Fc domain;
wherein the T cell engaging immune effector domain is linked to the N terminus of VC1, VC1 is linked via its C terminus to the N terminus of the FC1 region, the FC1 region is linked via its C terminus to the N terminus of VC2, and VC2 is linked via its C terminus to the N terminus of the FC2 region; and
wherein the pMHC binding domain and the T cell engaging immune effector domain are capable of binding to a pMHC complex and a T cell respectively.

5. The multi-domain binding molecule of claim 1, wherein the T cell engaging immune effector is an ScFv.

6. The multi-domain binding molecule of any preceding claim, wherein
(i) VC1 comprises either (a) a TCRα variable and constant region or (b) a TCRβ variable and constant region, and
(ii) VC2 comprises the other of (a) and (b).

7. The multi-domain binding molecule of claim 6, wherein VC1 comprises the TCRβ variable and constant region and VC2 comprises the TCRα variable and constant region.

8. The multi-domain binding molecule of any one of claims 1 to 5, wherein
(i) VC1 comprises either (a) a heavy chain antibody variable region or (b) a light chain antibody variable region, and
(ii) VC2 comprises the other of (a) and (b).

9. The multi-domain binding molecule of claim 4 or any one of claims 5 to 8 when appended to claim 4, wherein the TCE-VL region is linked via its C terminus to the N terminus of the TCE-VH region and the TCE-VH region is linked via its C terminus to the N terminus of VC1.

10. The multi-domain binding molecule of any preceding claim, wherein the immune cell engaging domain or the T cell engaging immune effector domain is a CD3 effector domain that activates a T cell through interaction with CD3 and/or a TCR/CD3 complex.

11. The multi-domain binding molecule of any preceding claim, wherein the immune cell engaging domain or the T cell engaging immune effector domain is an anti-CD3 scFv.

12. The multi-domain binding molecule of any preceding claim, wherein the pMHC binding domain binds to a tumour associated antigen peptide in complex with an MHC.

13. The multi-domain binding molecule of any preceding claim, wherein two or more of the ICE, TCE-VH, TCE-VL, VC1, VC2, FC1 and FC2 regions are linked to each other via linkers and/or IgG hinge sequences.

14. The multi-domain binding molecule of claim 13, wherein the linker or linkers have a sequence selected from the group of GGGGS (SEQ ID NO: 18), GGGSG (SEQ ID NO: 20), GGSGG (SEQ ID NO: 21), GSGGG (SEQ ID NO: 22), GSGGGP (SEQ ID NO: 23), GGEPS (SEQ ID NO: 24), GGEGGGP (SEQ ID NO: 25), GGEGGGSEGGGS (SEQ ID NO: 26), GGGSGGGG (SEQ ID NO: 47), GGGGSGGGGSGGGGSGGGGSGGGS (SEQ ID NO: 39), GGGGSGGGGSGGGGS (SEQ ID NO: 49), EAAAK (SEQ ID NO: 50) and EAAAKEAAAKEAAAK (SEQ ID NO: 51).

15. The multi-domain binding molecule of any preceding claim, wherein VC1 is linked to the FC1 region via a sequence comprising an IgG hinge sequence and/or VC2 is linked to the FC2 region via a sequence comprising an IgG hinge sequence.

16. The multi-domain binding molecule of claim 15, wherein the IgG hinge sequence is at least 80% identical to SEQ ID NO: 44.

17. The multi-domain binding molecule of claim 15 or claim 16, wherein the sequence linking VC1 to the FC1 region further comprises a glycine-serine linker and/or the sequence linking VC2 to the FC2 region further comprises a glycine-serine linker.

18. The multi-domain binding molecule of claim 17, wherein the glycine-serine linker has the sequence provided in SEQ ID NO: 47.

19. The multi-domain binding molecule of claim 4 or any one of claims 5 to 18 when appended to claim 4, wherein the TCE-VL region is linked to the TCE-VH region via a sequence comprising a glycine-serine linker, optionally wherein the sequence is provided in SEQ ID NO: 39.

20. The multi-domain binding molecule of claim 4 or any one of claims 5 to 19 when appended to claim 4, wherein the TCE-VH region is linked to VC1 via a sequence comprising a glycine-serine linker, optionally wherein the sequence is provided in SEQ ID NO: 18.

21. The multi-domain binding molecule of any preceding claim, wherein the FC1 region is linked to VC2 via a sequence comprising a glycine-serine linker, optionally wherein the sequence is provided in SEQ ID NO: 47.

22. The multi-domain binding molecule of any preceding claim, wherein the half-life extending domain comprises one or more amino acid substitutions which facilitate dimerisation of the FC1 region and the FC2 region.

23. The multi-domain binding molecule of claim 22, wherein:
(i) one of the FC1 region and the FC2 region comprises one or more amino acid substitutions selected from the group consisting of T366S, L368A, T394S, F405A, Y407A, Y407T and Y407V, according to the EU numbering scheme; and
(ii) the other of the FC1 region and the FC2 region comprises one or more amino acid substitutions selected from the group consisting of T366W, T366Y, T366W, T394W and F405W, according to the EU numbering scheme.

24. The multi-domain binding molecule of claim 22, wherein:
(i) one of the FC1 region and the FC2 region comprises one or more amino acid substitutions selected from the group consisting of T366S, L368A, and Y407V, according to the EU numbering scheme; and
(i) the other of the FC1 region and the FC2 region comprises a T366W amino acid substitution, according to the EU numbering scheme.

25. The multi-domain binding molecule of any preceding claim, wherein the half-life extending domain comprises one or more amino acid substitutions which attenuate an effector function of the Fc domain.

26. The multi-domain binding molecule of claim 25, wherein the half-life extending domain comprises one or more amino acid substitutions selected from the group consisting of S228P, E233P, L234A, L235A, L235E, L235P, G236R, G237A, P238S, F241A, V264A D265A, H268A, D270A, N297A, N297G, N297Q, E318A, K322A, L328R, P329G, P329A, A330S, A330L, P331A and P331S, according to the EU numbering scheme.

27. The multi-domain binding molecule of claim 25 or claim 26, wherein the half-life extending domain comprises one or more amino acid substitutions which prevent or reduce binding to FcγR.

28. The multi-domain binding molecule of claim 27, wherein the FC1 region and/or the FC2 region comprise a N297G amino acid substitution, according to the EU numbering scheme.

29. The multi-domain binding molecule of any preceding claim, wherein the half-life extending domain comprises one or more amino acid substitutions which promote binding to FcRn.

30. The multi-domain binding molecule of claim 29, wherein the FC1 region and/or FC2 region comprise M252Y, S254T and T256E amino acid substitutions, numbered according to the EU numbering scheme.

31. The multi-domain binding molecule of claim 4, or any one of claims 5 to 30 when appended to claim 4, wherein the T cell engaging immune effector domain comprises:
(i) a VL region comprising CDRs of SEQ ID NO: 33, 34, and 35 as CDR1, CDR2 and CDR3 respectively; and
(ii) a VH region comprising CDRs of SEQ ID NO: 36, 37, and 38 as CDR1, CDR2 and CDR3 respectively.

32. The multi-domain binding molecule of claim 4, or any one of claims 5 to 31 when appended to claim 4, wherein the T cell engaging immune effector domain comprises a VL region at least 80% identical to the sequence of SEQ ID NO: 31 and a VH region at least 80% identical to the sequence of SEQ ID NO: 32.

33. The multi-domain binding molecule of any preceding claim, wherein the FC1 and FC2 regions are IgG1 Fc regions.

34. The multi-domain binding molecule of any preceding claim, wherein:
i) the FC1 region comprises an amino acid sequence that is at least 80% identical to the sequence of SEQ ID NO: 42 and the FC2 region comprises an amino acid sequence that is at least 80% identical to the sequence of SEQ ID NO: 43; or
ii) the FC1 region comprises an amino acid sequence that is at least 80% identical to the sequence of SEQ ID NO: 57 and the FC2 region comprises an amino acid sequence that is at least 80% identical to the sequence of SEQ ID NO: 58.

35. The multi-domain binding molecule of any preceding claim, which comprises the following amino acid sequences, in the following order, from N-terminus to C-terminus:
a) an amino acid sequence of an anti-CD3 scFv, optionally followed by a linker sequence provided in SEQ ID NO: 18;
b) an amino acid sequence of a TCRβ variable and constant region;
c) a linker sequence provided in SEQ ID NO: 47 followed by an IgG hinge sequence provided in SEQ ID NO: 44;
d) an Fc region having the sequence provided in SEQ ID NO: 42;
e) a linker sequence provided in SEQ ID NO: 47;
f) an amino acid sequence of a TCRα variable and constant region;
g) a linker sequence provided in SEQ ID NO: 47 followed by an IgG hinge sequence provided in SEQ ID NO: 44; and
h) an Fc region having the sequence provided in SEQ ID NO: 43.

36. A nucleic acid encoding the multi-domain binding molecule of any preceding claim.

37. An expression vector comprising the nucleic acid of claim 36.

38. A host cell comprising the nucleic acid of claim 36 or the expression vector of claim 37.

39. A method of making the multi-domain binding molecule of any one of claims 1 to 35, comprising maintaining the host cell of claim 38 under optimal conditions for expression of the nucleic acid of claim 36 or the expression vector of claim 37 and isolating the multi-domain binding molecule.

40. A pharmaceutical composition comprising the multi-domain binding molecule of any one of claims 1 to 35, the nucleic acid of claim 36, the expression vector of claim 37 or the host cell of claim 38.

41. The multi-domain binding molecule of any one of claims 1 to 35, the nucleic acid of claim 36, the expression vector of claim 37, the host cell of claim 38 or the pharmaceutical composition of claim 40, for use as a medicament.

42. A method of treatment comprising administering the binding molecule of any one of claims 1 to 35, the nucleic acid of claim 36, the expression vector of claim 37, the host cell of claim 38 or the pharmaceutical composition of claim 40, to a patient in need thereof.

43. The multi-domain binding molecule of any one of claims 1 to 35, the nucleic acid of claim 36, the expression vector of claim 37, the host cell of claim 38 or the pharmaceutical composition of claim 40, for use in treating cancer, an autoimmune disease or an infectious disease.

44. The multi-domain binding molecule, nucleic acid, expression vector, host cell or pharmaceutical composition of claim 43, wherein the cancer is associated with PRAME expression.


SEQUENCE LISTING (58 sequences)

SEQ ID NO: 1 - protein, 9 aa, Homo sapiens
SLLQHLIGL

SEQ ID NO: 2 - protein, 115 aa, synthetic construct
GDAKTTQPNSMESNEEEPVHLPCQHSTISGTDYIHWYRQLPSQGPEYVIHGLTSNVNNRMASLAIAEDRKSSTLILHRATLRDAAVYYCILILGHSRLGNYIATFGKGTKLSVIP

SEQ ID NO: 3 - protein, 7 aa, synthetic construct
TISGTDY

SEQ ID NO: 4 - protein, 5 aa, synthetic construct
GLTSN

SEQ ID NO: 5 - protein, 17 aa, synthetic construct
CILILGHSRLGNYIATF

SEQ ID NO: 6 - protein, 17 aa, synthetic construct
IHWYRQLPSQGPEYVIH

SEQ ID NO: 7 - protein, 33 aa, synthetic construct
VNNRMASLAIAEDRKSSTLILHRATLRDAAVYY

SEQ ID NO: 8 - protein, 114 aa, synthetic construct
DGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVT

SEQ ID NO: 9 - protein, 5 aa, synthetic construct
LNHDA

SEQ ID NO: 10 - protein, 7 aa, synthetic construct
SQIMGDE

SEQ ID NO: 11 - protein, 15 aa, synthetic construct
CASSWWTGGASPIRF

SEQ ID NO: 12 - protein, 17 aa, synthetic construct
MYWYRQDPGQGLRLIYY

SEQ ID NO: 13 - protein, 35 aa, synthetic construct
QKGDIAEGYSVSREKKESFPLTVTSAQKNPTAFYL

SEQ ID NO: 14 - protein, 201 aa, synthetic construct
GDAKTTQPNSMESNEEEPVHLPCQHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 15 - protein, 86 aa, synthetic construct
NIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 16 - protein, 244 aa, synthetic construct
DGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 17 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 18 - protein, 5 aa, synthetic construct
GGGGS

SEQ ID NO: 19 - protein, 130 aa, synthetic construct
EDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 20 - protein, 5 aa, synthetic construct
GGGSG

SEQ ID NO: 21 - protein, 5 aa, synthetic construct
GGSGG

SEQ ID NO: 22 - protein, 5 aa, synthetic construct
GSGGG

SEQ ID NO: 23 - protein, 6 aa, synthetic construct
GSGGGP

SEQ ID NO: 24 - protein, 5 aa, synthetic construct
GGEPS

SEQ ID NO: 25 - protein, 7 aa, synthetic construct
GGEGGGP

SEQ ID NO: 26 - protein, 12 aa, synthetic construct
GGEGGGSEGGGS

SEQ ID NO: 27 - protein, 26 aa, synthetic construct
GDAKTTQPNSMESNEEEPVHLPCQHS

SEQ ID NO: 28 - protein, 10 aa, synthetic construct
GKGTKLSVIP

SEQ ID NO: 29 - protein, 26 aa, synthetic construct
DGGITQSPKYLFRKEGQNVTLSCEQN

SEQ ID NO: 30 - protein, 9 aa, synthetic construct
GPGTRLTVT

SEQ ID NO: 31 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 32 - protein, 122 aa, synthetic construct [sequence omitted]

SEQ ID NO: 33 - protein, 6 aa, synthetic construct
QDIRNY

SEQ ID NO: 34 - skipped

SEQ ID NO: 35 - protein, 9 aa, synthetic construct
QQGNTLPWT

SEQ ID NO: 36 - protein, 8 aa, synthetic construct
GYSFTGYT

SEQ ID NO: 37 - protein, 8 aa, synthetic construct
INPYKGVS

SEQ ID NO: 38 - protein, 15 aa, synthetic construct
ARSGYYGDSDWYFDV

SEQ ID NO: 39 - protein, 24 aa, synthetic construct
GGGGSGGGGSGGGGSGGGGSGGGS

SEQ ID NO: 40 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSS

SEQ ID NO: 41 - protein, 122 aa, synthetic construct
EVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSS

SEQ ID NO: 42 - protein, 217 aa, synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 217 aa, synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 15 aa, synthetic construct
EPKSSDKTHTCPPCP

SEQ ID NO: 45 - protein, 1191 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRADGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGSGGGGGDAKTTQPNSMESNEEEPVHLPCQHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTQVSQSKDSDVYITDKCVLDMRSMDFKSNSAVAWSQKSDFACANAFQNSIIPEDTGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 1191 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRADGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGSGGGGGDAKTTQPNSMESNEEEPVHLPCQHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTQVSQSKDSDVYITDKCVLDMRSMDFKSNSAVAWSQKSDFACANAFQNSIIPEDTGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 8 aa, synthetic construct
GGGSGGGG

SEQ ID NO: 48 - protein, 8 aa, synthetic construct
GYSFTGYA

SEQ ID NO: 49 - protein, 15 aa, synthetic construct
GGGGSGGGGSGGGGS

SEQ ID NO: 50 - protein, 5 aa, synthetic construct
EAAAK

SEQ ID NO: 51 - protein, 15 aa, synthetic construct
EAAAKEAAAKEAAAK

SEQ ID NO: 52 - protein, 10 aa, synthetic construct
DKTHTCPPCP

SEQ ID NO: 53 - protein, 12 aa, synthetic construct
ESKYGPPCPSCP

SEQ ID NO: 54 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 1187 aa, Synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSEAGVAQSPRYKIIEKRQSVAFWCNPISGHGTLYWYQQILGQGPKLLIQFHENGVVDDSQLPKDRFSAERLKGVDSTLKIQPAKLEDSAVYLCASSVDWVGDGERQYFGPGTRLLVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRADGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGSGGGGLAKTTQPISMDSYEGQEVNITCSHNYIAANDFITWYQQFPSQGPRFFIQGYKTNVQNEVASLFIPADRKSSTLSLPRVSLSDTAVYYCLAWGGTDLLPFGTGTRLQVFPNIQNPDPAVYQLRDSKSSDKSVCLFTDFDSQTQVSQSKDSDVYITDKCVLDMRSMDFKSNSAVAWSQKSDFACANAFQNSIIPEDTGGGSGGGGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 9 aa, Homo sapiens
SLSNRLYYL

SEQ ID NO: 57 - protein, 217 aa, synthetic construct
APELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 217 aa, synthetic construct
APELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK